(19) United States
(12) Patent Application Publication
(10) Pub. No.: US 2019/0254997 A1
(43) Pub. Date: Aug. 22, 2019

(54) METHODS FOR INDUCING STEREOCILIA ON HAIR CELLS

(71) Applicant: The Regents of the University of California, Oakland, CA (US)

(72) Inventors: Allen F. Ryan, Solana Beach, CA (US); Akiko Taura, Kyoto (JP)

(21) Appl. No.: 16/318,689

(22) PCT Filed: Jul. 20, 2017
(86) PCT No.: PCT/US17/43075

§ 371 Date: Jan. 17, 2019

Related U.S. Application Data
(60) Provisional application No. 62/365,189, filed on Jul. 21, 2016.

(51) Int. Cl. A61K 31/166; C12N 15/86; A61K 9/127; A61K 9/51; A61K 9/00; A61P 27/16

(52) U.S. Cl. CPC A61K 31/166; C12N 15/86; A61K 9/127; A61K 9/51; C12N 2750/14143; A61P 27/16; C12N 2710/10043; C12N 2740/10043; A61K 9/0046

(57) ABSTRACT

Provided are methods for inducing functional stereocilia and stereocilia bundles in inner ear auditory hair cells comprising administering to and/or expressing in the hair cells sufficient levels of espin isoform 1 (ESPN1). Further provided are otic solutions for delivering a polynucleotide encoding ESPN1 or a ESPN1 polypeptide to the inner ear of a subject.

### CROSS REFERENCE TO RELATED APPLICATIONS

This application claims the benefit of priority under 35 U.S.C. § 119(e) of U.S. Provisional Application No. 62/365,189, filed on Jul. 21, 2016, which is hereby incorporated herein in its entirety for all purposes.

### STATEMENT OF GOVERNMENTAL SUPPORT

This invention was made with government support under Grant No DC000129 awarded by the National Institutes of Health, and Grant No. BX001295 awarded by the United States Department of Veterans Affairs. The government has certain rights in the invention.

### BACKGROUND

Hearing and balance disorders are important causes of disability in patients. The major cause is sensory hair cell (HC) loss in the inner ear (1). In the damaged sensory epithelia of birds, new HCs regenerate in both the vestibular (2) and auditory sense organs (3). However, in mammals, HC loss is irreversible. For this reason, the regeneration of HCs in mammals is a subject of considerable research. Successful regeneration of functional HCs in mammals would be a major advance in the therapy of inner ear disorders.

When HCs are damaged, apical structures including stereociliary bundles are often the first cellular elements to be lost (4). The stereociliary bundle, as the site of mechanotransduction, is critical for HCs to maintain their physiological function. Some of these bundleless HCs can survive for considerable periods of time, but spontaneous stereociliary regeneration is not normally observed (5). So the replacement of stereocilia on surviving HCs is a potential strategy for functional recovery following inner ear sensory cell damage.

The espins are a family of actin bundling proteins, produced in multiple isoforms from a single gene (espn). They are localized in the stereocilia of HCs as well as the microvilli of many sensory cells (6). Espins are associated with the parallel actin bundles of stereocilia throughout stereocilia formation during development (7-8). Stereocilia are hypoplastic in the absence of the espn gene, exhibiting reduced length and abnormal structure. This results in hearing loss and vestibular dysfunction in the Jerker mouse model (9) and in the human DFNB36 deafness mutation (10). Given its critical role in stereociliary development, we reasoned that induced espin expression might be useful for stereociliary bundle regeneration.

Gene therapy has been found to be a promising tool for application to the inner ear. The labyrinth is anatomically well suited to local gene therapy, because it is a closed system that is isolated from other organs and is relatively easy to access via the middle ear, thus allowing local application and relative isolation of viral vectors with minimal spread to other sites (11). Recently, a degree of success has been reported using experimental gene therapy for various types of inner ear disorder caused by ototoxic drugs and genetic abnormalities (12-14).

With respect to HC regeneration, techniques which recapitulate the developmental program of HCs have generally been employed. These can, under the appropriate circumstances, induce nonsensory cells to convert to HCs with or without cell division (15-19). One of the earliest steps in HC specification is induction of the basic helix-loop-helix transcription factor ATOH1 by EYA1 and SIX1 (20). ATOH1 interacts with the Notch signaling system to form the mosaic of HCs and supporting cells, with Notch ligands suppressing the production of ATOH1 in the latter (21). The transduction inner ear supporting cells with the atoh1 gene has been reported to be effective in inducing HC formation, especially in developing mammals (22). Blockade of Notch signaling using γ-secretase inhibitors has also been shown to be effective in mammalian HC induction, since Notch signals act on supporting cells to inhibit their differentiation into HCs. Several studies have reported the conversion of supporting cells into HCs following Notch inhibition (23-25). The transdifferentiation process in the neonatal utricle is characterized by both mitotic and non-mitotic processes (21,26). Loss of HCs induces generally modest spontaneous regeneration due to loss of Notch inhibition of supporting cells. Notch inhibitors greatly enhance this process, inducing some supporting cells to dedifferentiate, enter the cell cycle, and produce daughter cells which can assume either a supporting cell or HC phenotype. Other supporting cells de-differentiate and transdifferentiate directly into HCs. However, in many cases regenerated HCs are immature and exhibit no or only partial function (23,25,27).

These prior studies suggest that enhancing the growth of stereocilia may be a useful means by which to restore cochlear function following HC damage and/or regeneration.

### SUMMARY

In one aspect, provided are methods of inducing stereocilia and/or stereociliary bundles in an inner ear auditory hair cell in a subject in need thereof. In varying embodiments, the methods comprise administering to and expressing within the hair cell in the inner ear of the subject a polynucleotide encoding espin isoform 1 (ESPN1), espin isoform 2 (ESPN2) and/or espin isoform 3 (ESPN3). In varying embodiments, the subject is a mammal, e.g., a human, a non-human primate, a canine, a feline, an equine or a rodent (a mouse, a rat, a hamster, a guinea pig, a rabbit). In a further aspect, provided are methods of inducing stereocilia and/or stereociliary bundles in an inner ear auditory hair cell comprising administering to and expressing within the hair cell a polynucleotide encoding espin isoform 1 (ESPN1), espin isoform 2 (ESPN2) and/or espin isoform 3 (ESPN3). In varying embodiments, the hair cell is in vivo. In varying embodiments, the hair cell is in vitro. In varying embodiments, the hair cell is a damaged or regenerated hair cell. In some embodiments, the hair cell has been damaged by exposure to an aminoglycoside, e.g., gentamicin. In varying embodiments, the polynucleotide encoding ESPN1, ESPN2 and/or ESPN3 is expressed under the control of a promoter heterologous to the polynucleotide, e.g., a constitutive promoter or an inducible promoter. In varying embodiments, the polynucleotide encoding ESPN1 is administered to the hair cell in a viral vector, e.g., a viral vector selected from the group consisting of adenovirus, adeno-associated virus, lentivirus, and retrovirus. In varying embodiments, the viral vector is replication defective. In varying embodiments, the polynucleotide encoding ESPN1 is administered to the hair cell in a plasmid vector. In some embodiments, the polynucleotide encoding ESPN1 has at least 60% sequence identity, e.g., at least about 65%, 70%, 75%, 80%, 85%, 90%, 95%, 95%, 97%, 98%, 99% or 100% sequence identity, to one or more of SEQ ID NOs: 1, 3, 5, 7 or 9. In varying embodiments, the methods further comprise administering to the hair cell a polynucleotide encoding ATOH1 (atonal bHLH transcription factor 1) or an ATOH1 polypeptide. In varying embodiments, the methods further comprising administering to the hair cell a Notch inhibitor, e.g., a γ-secretase inhibitor. In varying embodiments, the γ-secretase inhibitor is DAPT (N-[N-[(3,5-Difluorophenyl)acetyl]-L-alanyl]L-phenylglycinetert-butyl). In varying embodiments, the induced stereociliary bundles comprise a stair-step arrangement and a single, central kinocilium. In varying embodiments, the induced stereocilia in an induced stereociliary bundle are linked at their tops. In varying embodiments, the induced stereociliary bundles exhibit functional mechanoelectrical transduction channels.

In a further aspect, provided are methods of inducing stereocilia and/or stereociliary bundles in an inner ear auditory hair cell in a subject in need thereof comprising administering to the hair cell in the inner ear an ESPN1, ESPN2 and/or ESPN3 polypeptide. In varying embodiments, the subject is a mammal, e.g., a human, a non-human primate, a canine, a feline, an equine or a rodent (a mouse, a rat, a hamster, a guinea pig, a rabbit). In another aspect, provided are methods of inducing stereocilia and/or stereociliary bundles in an inner ear auditory hair cell. In some embodiments, the methods comprise administering to the hair cell an ESPN1, ESPN2 and/or ESPN3 polypeptide. In varying embodiments, the hair cell is in vivo. In varying embodiments, the hair cell is in vitro. In varying embodiments, the hair cell is a damaged or regenerated hair cell. In varying embodiments, the hair cell has been damaged by exposure to an aminoglycoside, e.g., gentamicin. In varying embodiments, the ESPN1 polypeptide is administered in a nanoparticle, a liposome and/or a microparticle. In varying embodiments, the ESPN1 polypeptide has at least 60% sequence identity, e.g., at least about 65%, 70%, 75%, 80%, 85%, 90%, 95%, 95%, 97%, 98%, 99% or 100% sequence identity, to one or more of SEQ ID NOs: 2, 4, 6, 8 or 10. In some embodiments, the methods further comprise administering to the hair cell a polynucleotide encoding ATOH1 (atonal bHLH transcription factor 1) or an ATOH1 polypeptide. In varying embodiments, the methods further comprising administering to the hair cell a Notch inhibitor, e.g., a γ-secretase inhibitor. In varying embodiments, the γ-secretase inhibitor is DAPT (N-[N-[(3,5-Difluorophenyl)acetyl]-L-alanyl]-L-phenylglycinetert-butyl). In varying embodiments, the induced stereociliary bundles comprise a stair-step arrangement and a single, central kinocilium. In varying embodiments, the induced stereocilia in an induced stereociliary bundle are linked at their tops. In varying embodiments, the induced stereociliary bundles exhibit functional mechanoelectrical transduction channels.

In a further aspect, provided is an otic solution (e.g., a solution formulated for delivery to the inner ear) comprising a polynucleotide encoding espin isoform 1 (ESPN1), espin isoform 2 (ESPN2) and/or espin isoform 3 (ESPN3). In varying embodiments, the polynucleotide is in a viral vector or a plasmid vector, e.g., a viral vector selected from the group consisting of adenovirus, adeno-associated virus, lentivirus, and retrovirus. In varying embodiments, the viral vector is replication defective. In varying embodiments, the polynucleotide encoding ESPN1 has at least 60% sequence identity, e.g., at least about 65%, 70%, 75%, 80%, 85%, 90%, 95%, 95%, 97%, 98%, 99% or 100% sequence identity, to one or more of SEQ ID NOs: 1, 3, 5, 7 or 9.

In a further aspect, provided is an otic solution (e.g., a solution formulated for delivery to the inner ear) comprising an ESPN1, ESPN2 and/or ESPN3 polypeptide. In varying embodiments, the ESPN1 polypeptide has at least 60% sequence identity to one or more of SEQ ID NOs: 2, 4, 6, 8 or 10.

### DEFINITIONS

A “polynucleotide” is a single- or double-stranded polymer of deoxyribonucleotide or ribonucleotide bases read from the 5′ to the 3′ end. Polynucleotides include RNA and DNA, and may be isolated from natural sources, synthesized in vitro, or prepared from a combination of natural and synthetic molecules. Sizes of polynucleotides are expressed as base pairs (abbreviated “bp”), nucleotides (“nt”), or kilobases (“kb”). Where the context allows, the latter two terms may describe polynucleotides that are single-stranded or double-stranded. When the term is applied to double-stranded molecules it is used to denote overall length and will be understood to be equivalent to the term “base pairs”. It will be recognized by those skilled in the art that the two strands of a double-stranded polynucleotide may differ slightly in length and that the ends thereof may be staggered as a result of enzymatic cleavage; thus all nucleotides within a double-stranded polynucleotide molecule may not be paired.

The terms “identical” or percent “identity,” in the context of two or more nucleic acids or polypeptide sequences, refer to two or more sequences or subsequences that are the same or have a specified percentage of amino acid residues or nucleotides that are the same (i.e., share at least about 80% identity, for example, at least about 85%, 90%, 91%, 92%, 93%, 94%, 95%, 96%, 97%, 98%, 99% identity over a specified region to a reference sequence, e.g., an ESPN polynucleotide or polypeptide sequence as described herein, when compared and aligned for maximum correspondence over a comparison window, or designated region as measured using one of the following sequence comparison algorithms or by manual alignment and visual inspection. Such sequences are then said to be “substantially identical.” This definition also refers to the compliment of a test sequence. Preferably, the identity exists over a region that is at least about 25 amino acids or nucleotides in length, for example, over a region that is 50, 100, 200, 300, 400, 500, 600, 700, 800 amino acids or nucleotides in length, or over the full-length of a reference sequence.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins to ESPN nucleic acids and proteins, the BLAST 2.4.0+ algorithms and the default parameters are used.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., eds., Current Protocols in Molecular Biology (1995 supplement)). Examples of algorithms that are suitable for determining percent sequence identity and sequence similarity are the BLAST and BLAST 2.0 algorithms, which are described in Altschul et al., J. Mol. Biol. 215:403-410 (1990) and Altschul et al., Nucleic Acids Res. 25:3389-3402 (1977), respectively. Software for performing BLAST analyses is publicly available through the National Center for Biotechnology Information (on the worldwide web at ncbi.nlm.nih.gov/).

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The terms “patient,” “subject,” “individual” interchangeably refer to a mammal, for example, a human or a non-human primate, a domesticated mammal (e.g., a canine or feline), an agricultural mammal (e.g., a bovine, porcine, ovine, equine), a laboratory mammal (a mouse, rat, hamster, guinea pig, rabbit).

The term “co-administer” refers to the simultaneous presence of two active agents in the blood of an individual. Active agents that are co-administered can be concurrently or sequentially delivered.

The phrase “cause to be administered” refers to the actions taken by a medical professional (e.g., a physician), or a person controlling medical care of a subject, that control and/or permit the administration of the agent(s)/compound(s) at issue to the subject. Causing to be administered can involve diagnosis and/or determination of an appropriate therapeutic or prophylactic regimen, and/or prescribing particular agent(s)/compounds for a subject. Such prescribing can include, for example, drafting a prescription form, annotating a medical record, and the like.

The terms “effective amount” or “amount effective to” or “therapeutically effective amount” includes reference to a dosage of a therapeutic agent sufficient to produce a desired result, such as inhibiting, reducing or preventing the disease condition sought to be inhibited, ameliorated and/or treated, e.g., hearing loss due to loss of functional stereocilia and/or stereociliary bundles on auditory hair cells, e.g., due to presbycusis (age-related), ototoxicity, noise induced hearing loss, viral infections of the inner ear, autoimmune inner ear diseases, genetics/heredity, inner ear barotrauma; physical trauma, surgical trauma, and/or inflammation. The term “effective amount” as used in relation to pharmaceutical compositions, typically refers to the amount of the active ingredient, e.g. the peptides of the invention, which are required to achieve the desired goal. For example, in therapeutic applications, an effective amount will be the amount required to be administered to a patient to result in treatment of the particular disorder for which treatment is sought (e.g., hearing loss due to loss of functional stereocilia and/or stereociliary bundles on auditory hair cells, e.g., due to presbycusis (age-related), ototoxicity, noise induced hearing loss, viral infections of the inner ear, autoimmune inner ear diseases, genetics/heredity, inner ear barotrauma; physical trauma, surgical trauma, and/or inflammation). The term “treatment of a disorder” denotes the reduction or elimination of symptoms of a particular disorder. Effective amounts will typically vary depending upon the nature of the disorder, the peptides used, the mode of administration, and the size and health of the patient. In one embodiment, the effective amount of the peptides of the invention ranges from 1 μg to 1 g of peptide for a 70 kg patient, and in one embodiment, from 1 μg to 10 mg. In one embodiment, the concentration ESPN polynucleotide or polypeptide administered ranges from 0.1 μM to 10 mM, and in one embodiment, from 5 μM to 1 mM, in one embodiment, from 5 μM to 100 μM, and in one embodiment from 5 μM to 40 μM.

As used herein, the term “treating” is intended to mean the administration of a therapeutically effective amount of an ESPN polynucleotide and/or ESPN polypeptide described herein to a subject who is experiencing loss or impairment of hearing, loss or impairment of balance, or injury to or loss of vestibular hair cells, neurons, supporting cells, or dark cells, in order to minimize, reduce, or completely prevent or restore, the loss of hearing, the loss of balance function or of hair cells, neurons or dark cells of the vestibular portion of the inner ear. Treatment is intended to also include the possibility of inducing, causing or facilitating regeneration of the cellular elements of the inner ear including hair cells, supporting cells, dark cells, neurons and subcellular organelles of these cells including, synapses, stereocilia bundles, kinocilia, mitochondria and other cell organelles, or mechanical and functional supporting structures such as otoconia, cupula and crista of the inner ear. Treatment is also intended to prevent recurrent degeneration after regeneration of cellular elements of the inner ear, including hair cells, supporting cells, dark cells, neurons and subcellular organelles of these cells including synapses, stereocilia bundles, kinocilia, mitochondria and other cell organelles, or mechanical and functional supporting structures such as otoconia, cupula and crista of the inner ear. Treatment is also intended to mean the partial or complete restoration of hearing or balance function regardless of the cellular mechanisms involved.

As used herein, “loss of balance” or “impairment to the sense balance”, “impaired balance”, “loss of balance function” and “balance disorders” are terms that are intended to refer to a deficit in the vestibular system including associated neural structures, or vestibular function of a subject compared to the system of a normally functioning human. This deficit may completely or partially impair a subject's ability to maintain posture, spatial orientation, locomotion and any other functions associated with normal vestibular function. Balance disorders also include intermittent attacks of vertigo, such as those seen in Meniere's Disease, or other inner ear disorders.

As used herein, the term “administration” is intended to include, but is not limited to, the following delivery methods: topical, including topical delivery to the round window membrane of the cochlea, oral, parenteral, subcutaneous, transdermal, and transbuccal administration. In one example, the permeability of the round window membrane is enhanced by partially digesting it using a protease prior to transfection of the inner ear cells with an ESPN polynucleotide and/or ESPN polypeptide described herein.

As used herein the term “hearing loss” is intended to mean any reduction in a subject's ability to detect sound. Hearing loss is defined as a 10 decibel (dB) standard threshold shift or greater in hearing sensitivity for two of 6 frequencies ranging from 0.5-6.0 (0.5, 1, 2, 3, 4, and 6) kHz (cited in Dobie, R. A. (2005) Audiometric Threshold Shift Definitions: Simulations and Suggestions, Ear and Hearing 26(1) 62-77). Hearing loss can also be only high frequency, and in this case would be defined as 5 dB hearing loss at two adjacent high frequencies (2-6 kHz), or 10 dB at any frequency above 2 kHz. One example of hearing loss is age-related (or aging-related) hearing loss, which is the gradual onset of hearing loss with increasing age.

As used herein, the term “prevention”, in the context of the loss of or impairments to the sense of balance, death or injury of vestibular hair cells, death or injury of vestibular neurons, injury to functionally important mechanical structures such as the ototoconia or cupula, death or injury of vestibular dark cells and the like refers to minimizing, reducing, or completely eliminating the loss or impairment of balance function or damage, death or loss of those cells through the administration of an effective amount of one of the vectors described herein, ideally before an oxidatively stressful insult, or less ideally, shortly thereafter.

### BRIEF DESCRIPTION OF THE DRAWINGS

FIGS. 1A-D illustrate the number of stereociliary bundles and HC bodies after GM treatment. A: control (myosin7A), B: control (phalloidin), C: GM 10d AT (myosin7A), D: GM 10d AT (phalloidin). After GM treatment, almost all hair bundles are lost, and the number of HC bodies decreases. However, some HCs without hair bundles remain even 10 days after damage (n=5 explants/time). Points represent means and bars represent SD (standard deviation).

FIGS. 2A-C illustrate adenoviral transduction after GM treatment. A: After Ad-GFP transduction, GFP expression was diffused throughout the cell body. B: After Ad-E1 transduction, a filamentous pattern of GFP expression was observed. C: After Ad-E4 transduction, a filamentous pattern of GFP expression was also observed. Most GFP positive cells were negative for Myosin7A (Red), suggesting that the majority of transduced cells were supporting cells. Red: Myosin7A, Green: EGFP, White: phalloidin, Blue: DAPI.

FIGS. 3A-C illustrate the effects of DAPT exposure after GM treatment. A, A′: control explant (2 days culture). HCs are densely present. B, B′: Two days after GM treatment. HCs decreased dramatically. C, C′: DAPT application after GM treatment. Many myosin7A-positive cells were observed. In a tissue section, myosin7A-positive cells were numerous in the supporting cell layer (arrows). A-C: whole mount. A′-C′: section. Red: Myosin7A, Blue: DAPI. HC*: HC layer SC**: supporting cell layer.

FIGS. 4A-C illustrate the time course of GFP expression after adenoviral transduction. A: Twenty-four hrs after Ad-E1 administration. B: Forty-eight hours after Ad-E1 administration. Filamentous GFP expression was observed (arrows). C: Intensity ratio of GFP expression for a typical explant. Adenoviral GFP expression is maximal 46 hrs after adenovirus administration.

FIGS. 5A-C illustrate the hair bundle-like structures on Ad-E1 transduced explants. A: Whole mount utricular explant. Many myosin7A-positive cells with hair-bundle-like structures on their apical surfaces are observed (e.g. arrows). B: Sectioned explant. Myosin7A-positive cell bodies are observed in the supporting cell layer. Hair-bundle-like structures protrude from extensions of these cells that reach the apical surface of the epithelium (e.g. arrowhead). Red: Myosin7A; Green: EGFP; Blue: DAPI. C: Number of hair-bundle-like structures observed after adenoviral transduction. In Ad-GFP transduced explants, a few damaged stereociliary bundles remained. However, in Ad-E1 transduced explants, there are many more immature hair bundles. In Ad-E4 transduced explants, some filamentous cilia-like structures are observed. The number of bundle-like structures in Ad-E1 transduced explants was significantly greater than in control (Ad-GFP) explants than in Ad-GFP transduced explants (p<0.05, 6-7 explants per group).

FIGS. 6A-D illustrate scanning electron microscopy (SEM) analysis. A-C: Representative explants. A: Ad-GFP transduction. Reticulated cell borders are observed. Some damaged hair bundles and basal bodies (e.g. arrowheads) remain, but few structures resembling stereocilliary arrays are present. B: Ad-E1 transduced explants. Many apparently immature hair-bundle-like structures are observed (e.g. arrows). C: Ad-E4. Extended microvillus-like structures and microvilli are apparent. There are some damaged streociliary bundles, but few immature hair-bundle-like structures are apparent. D. Quantitative analysis: Number of streociliary bundle-like structures in SEM. Significantly more of these structures are observed on Ad-E1 transduced explants, than on those transduced with Ad-GFP or Ad-E4 (P<0.001; 6-8 explants per treatment condition).

FIGS. 7A-B illustrate high magnification of Ad-E1 transduction in SEM observation. A: In each cell, one kinocilium is observed in the center of the cell and stereocilia are present on approximately half of the apical surface. B: Stereocilia exhibit a staircase pattern. The upper ends of the stereocilia appear to adhere to one another.

FIGS. 8A-D illustrate functional analysis of stereociliary bundles using FM1-43FX. A: Ad-GFP transduced explants. B: Ad-E1 transduced explants. C: Ad-E4 transduced explants. Only a few FM1-43 positive cells, as indicated by fluorescent labeling, are observed in A and C. In contrast, many more FM1-43 positive cells are seen in B (arrows). D: Quantitative FM1-43 analysis. In Ad-E1 transduced explants, there are a number of FM1-43-loaded cells. However, FM1-43 positive cells are much less prevalent in Ad-GFP and Ad-E4 explants. These differences were significant (P<0.05, 6-9 explants per treatment condition).

### DETAILED DESCRIPTION

### 1. Introduction

In the present study, we examined espn gene transduction to induce the regeneration of functional stereociliary arrays, following ototoxic HC damage and HC regeneration induced by Notch inhibition. We evaluated viral vector transduction with Espin1 or Espin4, both linked to GFP. A GFP-only vector was used as a control.

Once inner ear hair cells (HCs) are damaged by drugs, noise or aging, their apical structures including the stereociliary arrays are frequently the first cellular feature to be lost. While this can be followed by progressive loss of HC somata, a significant number of HC bodies often remain even after stereociliary loss. However, in the absence of stereocilia they are nonfunctional. HCs can sometimes be regenerated by Atoh1 transduction or Notch inhibition, but they also may lack stereociliary bundles. We therefore sought to develop methods for the regeneration of stereocilia, in order to achieve HC functional recovery. Espin is an actin bundling protein known to participate in sterociliary elongation during development. We evaluated stereociliary array regeneration in damaged vestibular sensory epithelia in tissue culture, using viral vector transduction of two espin isoforms. Utricular HCs were damaged with aminoglycosides. The utricles were then treated with a γ-secretase inhibitor, followed by espin or control transduction and histochemistry. While γ-secretase inhibition increased the number of HCs, few had stereociliary arrays. In contrast, 46 hrs after espin1 transduction, a significant increase in hair-bundle-like structures was observed. These were confirmed to be immature stereociliary arrays by scanning electron microscopy. Increased uptake of FM1-43 uptake provided evidence of stereociliary function. Espin4 transduction had no effect. The results demonstrate that espin1 gene therapy can restore stereocilia on damaged or regenerated HCs.

### 2. Subjects Amenable to Treatment

The methods described herein for inducing functional stereocilia and/or stereociliary bundles on inner ear auditory hair cells finds use for treating patients who have suffered hearing losses or who are likely to suffer hearing losses due to hair cell damage. In one example, the hearing loss is due to presbycusis (age-related hearing loss). In addition to age-related hearing loss, we also contemplate that other types of hearing loss may be treatable by administration to hair cells of ESPN1, ESPN2 and/or ESPN3, optionally co-administered with ATOH1. Examples of other types of hearing loss include, for example: 1) ototoxicity caused by chemical or pharmaceutical agents, for example, antineoplastic agents such as cisplatinum or related compounds, aminoglycosides, antineoplastic agents, and other chemical ototoxic agents; 2) noise induced hearing loss, either from acoustic trauma or blast injury; 3) therapeutic radiation; 4) viral infections of the inner ear, such as Herpes Simplex, cytomegalovirus or other viruses or infectious agents (such as Lyme Disease) that can cause inner ear hearing loss; 5) autoimmune inner ear diseases; 6) genetic hearing losses that may have an apoptotic component; 7) inner ear barotrauma such as diving or acute pressure changes; 8) physical trauma such as that caused by head injury, or surgical trauma from surgical intervention in the inner ear; 9) inflammation or other response to administration of other inner ear regenerative compounds or gene therapy techniques; 10) ischemic damage to the inner ear such as in vasculitis, or following acoustic neuroma surgery; and 11) idiopathic or vestibular disorders such as sudden sensurineural hearing loss.

### 3. Methods of Inducing Stereocilia in Hair Cells

Generally, the methods entail administering to an inner ear auditory hair cell a polynucleotide encoding Espin isoforms 1, 2 and/or 3, and/or Espin isoforms 1, 2 and/or 3 polypeptides. In some embodiments, ESPN1, ESPN2 and/or ESPN3 is co-administered with ATOH1, in polynucleotide or polypeptide form.

In general, there are two approaches to gene therapy in humans. For in vivo gene therapy, a vector encoding the gene of interest can be administered directly to the patient. Alternatively, in ex vivo gene therapy, cells are removed from the patient and treated with a vector to express the gene of interest. In the ex vivo method of gene therapy, the treated cells are then re-administered to the patient.

Numerous different methods for gene therapy are well known in the art. These methods include, but are not limited to, the use of DNA plasmid vectors as well as DNA and RNA viral vectors. In the present compositions and methods, these vectors are engineered to express ESPN isoforms 1, 2 and/or 3 (and optionally, ATOH1) when integrated into patient cells.

It is known that in the auditory system, three major viral vectors have been investigated for cochlear gene transfection: (1) lentivirus, (2) adenovirus and (3) Adeno-associated virus (AAV). The gene transfected by adenovirus vector has limited expression time and the vector has been associated with adverse immune reactions (Staecker, Brough, Praetorius, & Baker, 2004). The lentivirus vector, although capable of maintaining long term expression, is particularly suited for targeting neurons, but not hair cells (Federico, 1999). AAV vectors have several advantages such as long lasting expression of synthesized genes (Cooper et al, 2006), and low risk for pathogenic reactions (because they are artificially manufactured and not ototoxic) (Kaplitt et al., 1994). AAV vectors useful for targeting polynucleotides to the inner ear are described, e.g., in Shu, et al., Hum Gene Ther. (2016) 27(9):687-99 and Kilpatrick, et al., Gene Ther. (2011) 18(6):569-78.

Adenoviruses are able to transfect a wide variety of cell types, including non-dividing cells. The discovery includes the use of any one of more than 50 serotypes of adenoviruses that are known in the art, including the most commonly used serotypes for gene therapy: type 2 and type 5. In order to increase the efficacy of gene expression, and prevent the unintended spread of the virus, genetic modifications of adenoviruses have included the deletion of the E1 region, deletion of the E1 region along with deletion of either the E2 or E4 region, or deletion of the entire adenovirus genome except the cis-acting inverted terminal repeats and a packaging signal (Gardlik et al., Med Sci Monit. 11: RA110-121, 2005).

Adeno-associated virus (AAV) vectors can achieve latent infection of a broad range of cell types, exhibiting the desired characteristic of persistent expression of a therapeutic gene in a patient. The discovery includes the use of any appropriate type of adeno-associated virus known in the art including, but not limited to AAV1, AAV2, AAV3, AAV4, AAVS, AAV6 and AAV7 (Lee et al., Biochem J. 387: 1-15, 2005). Previous experiments have shown that genetic modification of the AAV capsid protein can be achieved to direct infection towards a particular tissue type (Lieber, Nature Biotechnology. 21: 1011-1013, 2003). Modified serotype-2 and -8 AAV vectors in which tyrosine residues in the viral envelope have been substituted for alanine residues that cannot be phosphorylated are also contemplated. In the case of tyrosine mutant serotype-2, tyrosine 444 is substitute with alanine (t2 mut 444). In the case of serotype 8, tyrosine 733 is substituted with an alanine reside (t8 mut 733). The titer for t2 mut 444 is 4.89E+12 and that for t8 mut 733 is 7.50E+13.

AAV vectors include those with a mutation of one or more surface-exposed tyrosine residues on capsid proteins. These mutated vectors avoid degradation by the proteasome, and significantly increase the transduction efficiency of these vectors. Mutation of one or more of the tyrosine residues on the outer surface of the capsid proteins including, for example, but not limited to, mutation of Tyr252 to Phe272 (Y252F), Tyr272 to Phe272 (Y272F), Tyr444 to Phe444 (Y444F), Tyr500 to Phe500 (Y500F), Tyr700 to Phe700 (Y700F), Tyr704 to Phe704), Tyr730 to Phe730 (Y730F) and Tyr733 to Phe733 (Y733F) provides improved transduction efficiency of the AAV vectors when compared to wild-type.

The modified vectors may facilitate penetration of the vector across the round window membranes, which would allow for non-invasive delivery of the vectors to the hair cells/spiral ganglion neurons of the cochlea. The EGFR-PTK (epidermal growth factor receptor-protein tyrosine kinase) phosphorylates tyrosine residues on the surface of the capsid targeting them for ubiquitinylation and degradation by the proteosome (Zhong, L, Zhao, W, Wu, J, Li, B, Zolotukhin, S, Govindasamy, L et al. (2007) A dual role of EGFR protein tyrosine kinase signaling in ubiquitination of AAV2 capsids and viral second-strand DNA synthesis. Mol Ther 15: 1323-1330). Using t2 mut 444 or t8 mut 733 it is possible to increase gene transfer by up to 10,000 fold decreasing the amount of AAV necessary to infect the sensory hair cells of the cochlea.

Using ex vivo gene therapy, an individual skilled in the art can be assured that the ESPN1, ESPN2 and/or ESPN3 (and optionally, ATOH1) protein or proteins will only be expressed in the desired tissue. In these applications, as well as applications where tissue specific expression of ESPN1, ESPN2 and/or ESPN3 (and optionally, ATOH1) is not a concern, the above vectors can be constructed to constitutively express ESPN1, ESPN2 and/or ESPN3 (and optionally, ATOH1) protein. Numerous constitutive regulator elements are well known in the art. Often, elements present in the native viruses described above are used to constitutively express a gene of interest. Other examples of constitutive regulatory elements include without limitation the chicken β-actin, EF1, EGR1, eIF4A1, FerH, FerL, GAPDH, GRP78, GRP94, HSP70, beta-Kin, ROSA, and ubiquitin B promoters.

For in vivo applications of gene therapy, the above vectors may be modified to include regulatory elements that confine the expression of ESPN1, ESPN2 and/or ESPN3 (and optionally, ATOH1) to certain tissue types. Numerous examples of regulatory elements specific to certain tissue types are well known in the art. Of particular interest to the discovery are elements that direct gene expression in the hair cells of the cochlea. An expression system for the inducible expression of ATOH1, that can find use for the inducible expression of ESPN1, ESPN2 and/or ESPN3 (and optionally, ATOH1) is described by Parker, et al., Hum Gene Ther Methods (2014) 25(1):1-13.

In some examples, it may be desirable to direct ESPN1, ESPN2 and/or ESPN3 (and ATOH1) expression in an inducible fashion. Several methods of inducible transgene expression are widely used. These methods consist of the transfection of the patient's cells with multiple viral or plasmid vectors. Typically, a first vector expresses the gene of interest under the control of a regulatory element that is responsive to the expression product of a second vector. The activity of this expression product is controlled by the addition of a pharmacological compound or some other exogenous stimulation. Examples of these systems are those that respond to tetracycline, mifepristone, ponasterone A, papamycin, tamoxifen, radiation, and heat shock (Robson et al., J. Biomed. Biotechnol. 2: 110-137, 2003).

Cochlear gene transfection in animals has utilized several approaches for vector delivery: (1) direct injection through round window membrane (RWM) into the perilymph, (2) intracochlear infusion through cochleostomy, and (3) transfusion through an intact RWM (Aarnisalo, et al., ORL J Otorhinolaryngol Relat Spec. (2006) 68(4):220-7). The third approach (transfusion through intact RWM) is least invasive and most likely to be accepted in human application.

The intact RWM consists of three layers: two epithelia layers separated by a layer of connective tissue, with collagen being a major component of the RWM. We have now demonstrated that the permeability of RWM can be increased temporarily by digestion of the membrane with collagenase. We then investigated whether (1) the digestion of RWM with the enzyme could facilitate the gene transfection of inner ear cells, and (2) if the digestion was safe to inner ear function and the structure of RWM. Instead of or in addition to enzymatic digestion of the RWM, proteidic delivery of polynucleotides across the RWM is described, e.g., by Qi, et al., Gene Ther. (2014) 21(1):10-8.

Additional approaches for delivery of a polynucleotide or polypeptide to the inner ear that can find use for delivering and ESPN polynucleotide or polypeptide include surgery facilitating viral delivery (Akil, et al., J V is Exp. (2015) Mar 16;(97)); nanoparticles and transtympanic injection (Zou, et al., J Nanobiotechnology. (2015) 13:5); dendrimer-based nanocarriers (Wu, et al., J Biomed Nanotechnol. (2013) 9(10):1736-45); and hyaluronic acid to enhance gene delivery (Shibata, et al., Hum Gene Ther. (2012) 23(3):302-10).

### 4. Formulation, Administration, Dosing and Scheduling

### a. Formulation and Administration of ESPN Polynucleotides

ESPN polynucleotides, e.g., in a viral or plasmid vector, can be incorporated into pharmaceutical compositions suitable for administration to a subject. In some particular embodiments, the pharmaceutical composition comprises the vectors described herein and a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In many cases, it can be preferable to include isotonic agents, for example, sugars, polyalcohols such as mannitol, sorbitol, or sodium chloride in the composition. Pharmaceutically acceptable carriers can further comprise minor amounts of auxiliary substances such as wetting or emulsifying agents, preservatives or buffers, which enhance the shelf life or effectiveness of the vector or pharmaceutical composition.

The compositions described herein may be in a variety of forms. These include, for example, liquid, semi-solid and solid dosage forms, such as liquid solutions (e.g., injectable and infusible solutions), dispersions or suspensions, tablets, pills, powders, liposomes and suppositories. The form used depends on the intended mode of administration and therapeutic application. Typical compositions are in the form of injectable or infusible solutions, such as compositions similar to those used for passive immunization of humans. The typical mode of administration is intratympanic (in the middle ear), intracochlear, parenteral (e.g., intravenous, subcutaneous, intraperitoneal, intramuscular, intrathecal). In one example, the vector is administered by intravenous infusion or injection. In another example, the vector is administered by intramuscular or subcutaneous injection. In another example, the vector is administered perorally. In yet another example, the vector is delivered to a specific location using stereostatic delivery, particularly through the tympanic membrane or mastoid into the middle ear.

Therapeutic compositions typically must be sterile and stable under the conditions of manufacture and storage. The composition can be formulated as a solution, microemulsion, dispersion, liposome, or other ordered structure suitable to high drug concentration. Sterile injectable solutions can be prepared by incorporating the vector in the required amount in an appropriate solvent with one or a combination of ingredients enumerated above, as required, followed by filtered sterilization.

Generally, dispersions are prepared by incorporating the vector into a sterile vehicle that contains a basic dispersion medium and the required other ingredients from those enumerated above. In the case of sterile lyophilized powders for the preparation of sterile injectable solutions, the preferred methods of preparation are vacuum drying and spray-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be achieved by including an agent in the composition that delays absorption, for example, monostearate salts and gelatin.

Therapeutic compositions typically must be sterile and stable under the conditions of manufacture and storage. The composition can be formulated as a solution, microemulsion, dispersion, liposome, or other ordered structure suitable to high drug concentration. Sterile injectable solutions can be prepared by incorporating the vector in the required amount in an appropriate solvent with one or a combination of ingredients enumerated above, as required, followed by filtered sterilization.

Generally, dispersions are prepared by incorporating the vector into a sterile vehicle that contains a basic dispersion medium and the required other ingredients from those enumerated above. In the case of sterile lyophilized powders for the preparation of sterile injectable solutions, the preferred methods of preparation are vacuum drying and spray-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be achieved by including an agent in the composition that delays absorption, for example, monostearate salts and gelatin.

The vector used with some embodiments as described herein can be incorporated into pharmaceutical compositions suitable for administration to a subject. In some particular embodiments, the pharmaceutical composition comprises the vectors described herein and a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In many cases, it can be preferable to include isotonic agents, for example, sugars, polyalcohols such as mannitol, sorbitol, or sodium chloride in the composition. Pharmaceutically acceptable carriers can further comprise minor amounts of auxiliary substances such as wetting or emulsifying agents, preservatives or buffers, which enhance the shelf life or effectiveness of the vector or pharmaceutical composition.

The compositions described herein may be in a variety of forms. These include, for example, liquid, semi-solid and solid dosage forms, such as liquid solutions (e.g., injectable and infusible solutions), dispersions or suspensions, tablets, pills, powders, liposomes and suppositories. The form used depends on the intended mode of administration and therapeutic application. Typical compositions are in the form of injectable or infusible solutions, such as compositions similar to those used for passive immunization of humans. The typical mode of administration is intratympanic (in the middle ear), intracochlear, parenteral (e.g., intravenous, subcutaneous, intraperitoneal, intramuscular, intrathecal). In one example, the vector is administered by intravenous infusion or injection. In another example, the vector is administered by intramuscular or subcutaneous injection. In another example, the vector is administered perorally. In yet another example, the vector is delivered to a specific location using stereostatic delivery, particularly through the tympanic membrane or mastoid into the middle ear.

The vectors described herein can be administered by a variety of methods known in the art. As will be appreciated by the skilled artisan, the route and/or mode of administration will vary depending upon the desired results. In certain embodiments, the vector may be prepared with a carrier that will protect the vector against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are generally known to those skilled in the art.

### b. Formulation and Administration of ESPN Polypeptides

Compositions comprising ESPN polypeptides (and optionally an ATOH1 polypeptide) for otic administration (e.g., administration into the ear) will commonly comprise a solution of the peptide comprising the peptide dissolved in a pharmaceutically acceptable carrier, preferably an aqueous carrier. A variety of aqueous carriers can be used, e.g., buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of the one or more peptides in these formulations can vary widely, and will be selected primarily based on fluid volumes, viscosities, body weight and the like in accordance with the particular mode of administration selected and the patient's needs.

Liquid form pharmaceutical preparations can include solutions, suspensions, and emulsions, for example, water or water/propylene glycol solutions. Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents. In varying embodiments, liquid preparations can be formulated in solution in aqueous polyethylene glycol solution.

In various embodiments, the therapeutic agent is encapsulated in a liposome coated with or conjugated to one or more ESPN polypeptides. Liposomes include emulsions, foams, micelles, insoluble monolayers, liquid crystals, phospholipid dispersions, lamellar layers and the like. In these preparations the composition of the invention to be delivered is incorporated as part of a liposome, alone or in conjunction with a molecule which binds to a desired target, such as antibody, or with other therapeutic or immunogenic compositions. Liposomes for use in the invention are formed from standard vesicle-forming lipids, which generally include neutral and negatively charged phospholipids and a sterol, such as cholesterol. The selection of lipids is generally guided by consideration of, e.g., liposome size, acid lability and stability of the liposomes in the blood stream. A variety of methods are available for preparing liposomes, as described in, e.g., Szoka, et al., Ann. Rev. Biophys. Bioeng. 9:467 (1980), U.S. Pat. Nos. 4,235,871; 4,501,728; 4,837,028 and 5,019,369. ESPN polypeptides can be integrated into, attached or conjugated directly to the liposome using methods known in the art.

In some embodiments, the ESPN polypeptides are attached or conjugated to a liposome or a nanoparticle that encapsulates the therapeutic agent. Nanoparticles for encapsulation and delivery of a therapeutic agent are known in the art and can find use. Illustrative nanoparticles include without limitation, e.g., semiconductor quantum dots (QDs), silicon (Si) nanoparticles (Park, et al., Nature Materials (2009) 8:331-336; Tu, et al., JACS, (2010) 132:2016-2023; Zhang, et al., JACS, (2007) 129:10668; Singh M P et al., ACS Nano, (2012) In press, (DOI: 10.1021/nn301536n); Rosso-Vasic, et al., J. Mater. Chem. (2009) 19:5926-5933; Bhattacharjee S., et al., Nanotoxicology, (2011) DOI 10.3109/17435390.2011.633714, 1 14; Chandra, et al., Nanoscale (2011) 3:1533-1540), polylactide-co-glycolide nanoparticles for controlled delivery of anticancer agents (Dinarvand, et al., Int J Nanomedicine. 2011;6:877-95); polyethyleneimine (PEI)-As(2)O(3)/Mn(0.5)Zn(0.5)Fe(2)O(4) magnetic nanoliposomes (Wang, et al., Int J Nanomedicine. 2011;6:871-5); redox-responsive poly(ethylene glycol)-b-poly(lactic acid) (MPEG-SS-PLA) nanoparticles (Song, et al., Colloids Surf B Biointerfaces. 2011, PMID 21719259); Thiolated Pluronic (Plu-SH) nanoparticles (Abdullah-Al-Nahain, et al., Macromol Biosci. 2011, PMID 21717576); and mesoporous silica nanoparticles (MSNs) (Wu, et al., Chem Commun (Camb). 2011, PMID 21716992). In one embodiment, the ESPN polypeptides are conjugated to biocompatible nanomicelles comprised of cholic acid, lysine and polyethylene glycol (PEG) covalently conjugated together, e.g., described in Xiao, et al., Biomaterials (2009) 30:6006-6016; and Luo, et al., Bioconjug Chem (2010) 21:1216-1224. Further nanomicelles that find use are described, e.g., in PCT Patent Publ. WO 2010/039496.

In one embodiment, ESPN polypeptides are attached or conjugated to biocompatible nanomicelles comprised of cholic acid, lysine and polyethylene glycol (PEG) covalently conjugated together, e.g., as described in Xiao, et al., Biomaterials (2009) 30:6006-6016; and Luo, et al., Bioconjug Chem (2010) 21:1216-1224. Recently, a biocompatible nanomicelle drug delivery system comprised of a unique amphiphilic polymers called telodendrimers was developed [Xiao, et al., Biomaterials (2009) 30:6006-6016; Luo, et al., Bioconjug Chem (2010) 21:1216-1224]. Telodendrimers consist of cholic acid, lysine and polyethylene glycol (PEG) covalently conjugated together, which impart the ability to self-assemble into a water-soluble spheroid with a hydrophobic core capable of sequestering many types of drugs. Cholic acid, a primary component of bile acid, possesses a facial amphiphilic structure: a rigid steroid scaffold with four hydrophilic groups on one surface, and hydrophobic methyl groups on the other surface of the scaffold. Lysine is a natural amino acid. PEG is biocompatible and has been used to improve the pharmacokinetics of therapeutic drugs. This nanocarrier system has many attractive characteristics for drug delivery, such as high drug loading capacity, narrow polydispersity, well-defined structure, easy chemical modification, superior physical, chemical stability and biocompatibility.

In some embodiments, the ESPN polypeptides in conjunction with a therapeutic agent is formulated as a nanoparticle. Nanoparticle conjugates are known in the art and described, e.g., in Musacchio, et al., Front Biosci. (2011) 16:1388-412; Cuong, et al., Curr Cancer Drug Targets. (2011) 11(2):147-55; Jain, BMC Med. (2010) 8:83; Sunderland, et al., Drug Development Research (2006) 67(1):70-93; Gu, et al., Nanotoday (2007) 2(3):14-21; Alexis, et al., ChemMedChem. (2008) 3(12):1839-43; Fay, et al., Immunotherapy. (2011) 3(3):381-394; Minko, et al., Methods Mol Biol. (2010) 624:281-94; and PCT Publ. Nos. WO 2011/046842; WO 2010/040062; WO 2010/047765; and WO 2010/120385, the disclosures of which are hereby incorporated herein by reference in their entirety for all purposes. Known nanoparticle cores find use in encapsulating a therapeutic agent (e.g., an ESPN polynucleotide or polypeptide) for delivering to a tissue of interest, e.g., an inner ear auditory hair cell. One or more ESPN polypeptides can be integrated into, attached or conjugated directly to the nanoparticle core using methods known in the art. In some embodiments, the encapsulating nanoparticle is a cylindrical PRINT nanoparticle, e.g., as described in Gratton, et al., Proc Natl Acad Sci USA. (2008) 105(33):11613-8. The nanoparticle can be biodegradable or non-biodegradable, as appropriate or desired. Poly(lactic acid-co-glycolic acid) (PLGA), biodegradable poly(L lactic acid) (PLLA) and PEG-based hydrogels find use as a matrix material in particle drug delivery systems because they are biocompatible, bioabsorbable, and have already shown promise in medical applications.

Peptide nanoparticles and methods for their preparation are known in the art and described, e.g., in U.S. Patent Publication No. 2006/0251726, U.S. Patent Publication No. 2004/0126900, U.S. Patent Publication No. 2005/0112089, U.S. Patent Publication No. 2010/0172943, U.S. Patent Publication No. 2010/0055189, U.S. Patent Publication No. 2009/0306335, U.S. Patent Publication No. 2009/0156480, and U.S. Patent Publication No. 2008/0213377, each of which is hereby incorporated herein by reference in its entirety for all purposes. Further nanoparticle formulations that find use are described, e.g., in Emerich and Thanos, Curr Opin Mol Ther (2008) 10(2):132-9; Kogan, et al., Nanomedicine (2007) 2(3):287-306; Zhang, et al., Bioconjug Chem (2008) 19(1):145-152; Scarberry, et al., J Am Chem Soc (2008) 130(31):10258-10262; Fraysse-Ailhas, et al., Eur Cells Materials (2007) 14(Suppl. 3):115; Corrias F, Lai F., Recent Pat Drug Deliv Formul. 2011 Aug. 12, PMID:21834772; Wang, et al., Biomaterials. (2011) 32(32):8281-90; and Kaur, et al., Artif Cells Blood Substit Immobil Biotechnol. 2011 Aug. 2., PMID:21806501. As appropriate, amino acid sequences may be added to either or both the N-terminus and the C-terminus of the peptide ligands in order to allow assembly and formation of the peptide nanoparticle.

### c. Dosing

The pharmaceutical formulation is preferably in unit dosage form. In such form the preparation is subdivided into unit doses containing appropriate quantities of the active component. The unit dosage form can be a packaged preparation, the package containing discrete quantities of preparation, such as packeted predetermined fluid volumes in vials or ampoules.

The term “unit dosage form”, as used in the specification, refers to physically discrete units suitable as unitary dosages for human subjects and animals, each unit containing a predetermined quantity of active material calculated to produce the desired pharmaceutical effect in association with the required pharmaceutical diluent, carrier or vehicle. The specifications for the novel unit dosage forms of this invention are dictated by and directly dependent on (a) the unique characteristics of the active material and the particular effect to be achieved and (b) the limitations inherent in the art of compounding such an active material for use in humans and animals, as disclosed in detail in this specification, these being features of the present invention.

The pharmaceutical compositions described herein can include a “therapeutically effective amount” or a “prophylactically effective amount” of the vectors described herein. A “therapeutically effective amount” refers to an amount effective, at dosages and for periods of time necessary, to achieve the desired therapeutic result, in this case for both prophylaxis and treatment of hearing loss or impairment of balance without unacceptable toxicity or undesirable side effects.

A therapeutically effective amount of the vector can vary according to factors such as the disease state, age, sex, and weight of the individual, and the ability of the vector to elicit a desired response in the individual. A therapeutically effective amount can also be one in which any toxic or detrimental effects of the vector are outweighed by the therapeutically beneficial effects. A “prophylactically effective amount” refers to an amount effective, at dosages and for periods of time necessary, to achieve the desired prophylactic result. Typically, since a prophylactic dose can be used in subjects prior to or at an earlier stage of disease, the prophylactically effective amount can be less than the therapeutically effective amount.

Dosage regimens can be adjusted to provide the optimum desired response (e.g., a therapeutic or prophylactic response). For example, a single bolus can be administered, several divided doses can be administered over time or the dose can be proportionally reduced or increased as indicated by the exigencies of the therapeutic situation. It can be especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of vector calculated to produce the desired therapeutic effect in association with the required pharmaceutical carrier. The specification for the dosage unit forms can be dictated by and directly dependent on (a) the unique characteristics of the vector and the particular therapeutic or prophylactic effect to be achieved, and (b) the limitations inherent in the art of formulating such vector for treating or preventing hearing loss or impaired balance in a subject.

Optimum dosages, toxicity, and therapeutic efficacy of compositions can further vary depending on the relative potency of individual compositions and can be determined by standard pharmaceutical procedures in cell cultures or experimental animals, for example, by determining the LD50 (the dose lethal to 50% of the population) and the ED50 (the dose therapeutically effective in 50% of the population). The dose ratio between toxic and therapeutic effects is the therapeutic index and can be expressed as the ratio, LD50/ED50. Compositions that exhibit large therapeutic indices are preferred. While compositions that exhibit toxic side effects can be used, care should be taken to design a delivery system that targets such compositions to the site of affected tissue to minimize potential damage to normal cells and, thereby, reduce side effects.

The data obtained from, for example, animal studies (e.g., rodents and monkeys) can be used to formulate a dosage range for use in humans. The dosage of conjugated ESPN polypeptide lies preferably within a range of circulating concentrations that include the ED50 with little or no toxicity. The dosage can vary within this range depending upon the dosage form employed and the route of administration. For any composition for use in the methods of the invention, the therapeutically effective dose can be estimated initially from cell culture assays. A dose can be formulated in animal models to achieve a circulating plasma concentration range that includes the IC50 (the concentration of the test compound that achieves a half-maximal inhibition of symptoms) as determined in cell culture. Such information can be used to more accurately determine useful doses in humans. Levels in plasma can be measured, for example, by high performance liquid chromatography (HPLC).

Following successful treatment, it may be desirable to have the subject undergo maintenance therapy to prevent the recurrence of the disease or malignant condition treated.

### d. Scheduling

Dosing schedules can be calculated from measurements of peptides in the body of a subject. In general, dosage is from 1 ng to 1,000 mg per kg of body weight and may be given once or more daily, semiweekly, weekly, biweekly, semimonthly, monthly, bimonthly or yearly, as needed or appropriate. Persons of ordinary skill in the art can easily determine optimum dosages, dosing methodologies and repetition rates. One of skill in the art will be able to determine optimal dosing for administration of a peptide or peptide composition of the present invention to a human being following established protocols known in the art and the disclosure herein.

Single or multiple administrations of the pharmaceutical formulations may be administered depending on the dosage and frequency as required and tolerated by the patient. In any event, the composition should provide a sufficient quantity of the peptides of this invention to effectively treat the patient. Preferably, the dosage is administered once but may be applied periodically until either a therapeutic result is achieved or until side effects warrant discontinuation of therapy. Generally, the dose is sufficient to treat or ameliorate symptoms or signs of disease without producing unacceptable toxicity to the patient.

The daily dose can be administered once per day or divided into subdoses and administered in multiple doses, e.g., twice, three times, or four times per day. However, as will be appreciated by a skilled artisan, compositions described herein may be administered in different amounts and at different times. The skilled artisan will also appreciate that certain factors may influence the dosage and timing required to effectively treat a subject, including but not limited to the severity of the disease or malignant condition, previous treatments, the general health and/or age of the subject, and other diseases present. Moreover, treatment of a subject with a therapeutically effective amount of a composition can include a single treatment or, preferably, can include a series of treatments.

Thus, a pharmaceutical formulation thereof for intratympanic (in the middle ear) or intracochlear or inner ear administration would be about 0.01 to 100 mg/kg per patient per day. Dosages from 0.1 up to about 1000 mg/kg per patient per day may be used, particularly when the drug is administered to a secluded site and not into the blood stream, such as into a body cavity or into a lumen of an organ. Actual methods for preparing parenterally administrable compositions will be known or apparent to those skilled in the art and are described in more detail in such publications as Remington: The Science and Practice of Pharmacy, 21st Ed., 2006, Lippincott Williams & Wilkins.

To achieve the desired therapeutic effect, pharmaceutical formulations may be administered for multiple days at the therapeutically effective daily dose. Thus, therapeutically effective administration of compositions to treat a disease or malignant condition described herein in a subject may require periodic (e.g., daily) administration that continues for a period ranging from three days to two weeks or longer. In varying embodiments, compositions can be administered for at least three consecutive days, often for at least five consecutive days, more often for at least ten, and sometimes for 20, 30, 40 or more consecutive days, or longer, as needed. While consecutive daily doses are a preferred route to achieve a therapeutically effective dose, a therapeutically beneficial effect can be achieved even if the compounds or compositions are not administered daily, so long as the administration is repeated frequently enough to maintain a therapeutically effective concentration of the composition in the subject. For example, one can administer a composition every other day, every third day, or, if higher dose ranges are employed and tolerated by the subject, once a week.

### 5. Methods of Monitoring

A variety of methods can be employed in determining efficacy of therapeutic and prophylactic treatments with the ESPN polynucleotides and/or ESPN polypeptides described herein. Generally, efficacy is the capacity to produce an effect without significant toxicity. Efficacy indicates that the therapy provides therapeutic or prophylactic effects for a given intervention (examples of interventions can include by are not limited to administration of a pharmaceutical formulation, employment of a medical device, or employment of a surgical procedure). Efficacy can be measured by comparing treated to untreated individuals or by comparing the same individual before and after treatment. Efficacy of a treatment can be determined using a variety of methods, including pharmacological studies, diagnostic studies, predictive studies and prognostic studies.

The methods of the present invention provide for improving hearing and inner ear hair cell function and/or reducing hearing loss and inner ear hair cell loss/destruction in a patient suffering from or susceptible to various middle ear and/or inner ear disorders, as described herein. A variety of methods can be used to monitor both therapeutic treatment for symptomatic patients and prophylactic treatment for asymptomatic patients.

Monitoring methods can entail determining a baseline value of a monitored parameter (e.g., general ability to hear, ability to hear high frequency sounds, hair cell structure and function) in a patient before administering a dosage of the ESPN polynucleotides and/or ESPN polypeptides, and comparing this with a value for the same parameter after treatment, respectively.

With respect to therapies using the ESPN polynucleotides and/or ESPN polypeptides, a significant decrease (e.g., greater than the typical margin of experimental error in repeat measurements of the same sample, expressed as one standard deviation from the mean of such measurements) in value of the measured parameter (e.g., hearing, hair cell function) signals a positive treatment outcome (e.g., that administration of ESPN polynucleotides and/or ESPN polypeptides has blocked or inhibited, or reduced progression of the disease condition sought to be treated). In some embodiments, treatment with the ESPN polynucleotides and/or ESPN polypeptides is considered to be efficacious if the measured parameter in the subject being treated is reduced by at least about 10%, for example, by at least about 20%, 30%, 40% or 50%, or by completely eliminating the symptoms of the measured parameter, e.g., comparing before and after treatment in the subject.

In other methods, a patient who is not presently receiving treatment but has undergone a previous course of treatment is monitored for one or more parameters or symptoms of middle ear or inner ear disease to determine whether a resumption of treatment is required. The measured value of the symptom or parameter in the patient can be compared with a value of the same symptom or parameter previously achieved in the patient after a previous course of treatment. A significant decrease in the symptom or parameter relative to the previous measurement (e.g., greater than a typical margin of error in repeat measurements of the same sample) is an indication that treatment need not be resumed. Alternatively, the value measured in a patient can be compared with a control value (mean plus standard deviation) determined in a population of patients after undergoing a course of treatment. Alternatively, the measured value in a patient can be compared with a control value in populations of prophylactically treated patients who remain free of symptoms of disease, or populations of therapeutically treated patients who show amelioration of disease characteristics. In all of these cases, a significant increase the symptoms or measured parameters relative to the control level (e.g., more than a standard deviation) is an indicator that treatment should be resumed in a patient.

### EXAMPLES

The following examples are offered to illustrate, but not to limit the claimed invention.

### Example 1

### Hair Cell Stereociliary Bundle Regeneration By Espin Gene Transduction After Aminoglycoside Damage And Hair Cell Induction By Notch Inhibition

The following example is published in Taura, et al., Gene Ther. (2016) 23(5):415-23, which is hereby incorporated herein by reference in its entirety for all purposes.

### Materials and Methods

Animals. CD-1 mice of both sexes were purchased from Japan SLC Inc., Hamamatsu, Japan and bred under standard husbandry conditions. They were housed under SPF conditions in rodent boxes on sawdust bedding. Pups were used at 2 days after birth. Experimental protocols were approved by the Animal Research Committee of Kyoto University Graduate School of Medicine and complied with the US National Institutes of Health Guidelines for the Care and Use of Laboratory Animals.

Production of Ad-espin vectors. Replication-deficient recombinant adenoviruses with deleted E1, E3, and E4 regions 18 were used. Expression plasmids containing cDNAs for the Espinl-EGFP conjugate (Espinl-EGFP) and the Espin4-EGFP conjugate were kindly provided by Prof. James Bartles of Northwestern University. Adenovirus Espinl-EGFP (Ad-E1) and adenovirus Espin4-EGFP (Ad-E4) were constructed using the Adenovirus Expression Vector Kit Ver. 2 (Takara) according to the manufacturer's protocol. In brief, the Espin1 or Espin4 cDNAs were transferred to a cosmid shuttle vector (pAxCAwtit, Takara) containing the human cytomegalovirus promoter and SV40 termination sequence, and amplified. Then they were linearized by restriction enzyme digest. The expression inserts were transferred to the pAdEasy-1 adenoviral vector35 by electroporation. The adenoviruses vectors were amplified in human embryonic kidney cells (HEK-293, RIKEN Bioresource Center, Cell No. RCB1637) and purified with the Adeno-X Maxi purification Kit (Clontech). We employed the vectors at a concentration of 1×10⁷ total particles of purified virus per milliliter (pfu/ml). Viral suspensions were kept at −80° C. until thawed for use. Appropriate protein production by Ad.E1-EGFP and Ad.E4-EGFP was confirmed by Western blotting following transduction of fibroblasts.

Dissection of vestibular maculae and tissue culture. Postnatal mice were decapitated under deep anesthesia and their temporal bones were removed. Utricula maculae were dissected from the surrounding tissue in 0.01M phosphate-buffered saline, pH 7.4. The otoconial membranes were gently removed with a fine needle. Explants of utricular sensory epithelia were placed intact on type I collagen-coated cover glasses (Iwaki, Tokyo, Japan) and maintained in 24-well culture plates (Iwaki) in Dulbecco's modified Eagle's medium (Invitrogen, Eugene, Oreg., USA), supplemented with 6 g/l glucose (Wako Pure Chemicals, Osaka, Japan) and 1.5 g/l penicillin G (Wako Pure Chemicals), at 37° C. in a humidified atmosphere of 95% air and 5% CO₂ for 24 hrs.

In order to examine the toxicity of gentamicin (GM) to HC stereociliary bundles and HC bodies, we damaged explants with 1mM GM (Nakarai, Japan) for 48 hrs and then maintained the explants in culture without GM for 10 days. After fixation with 4% paraformaldehyde and histochemistry (see below), we counted stereociliary bundles and HC bodies. Bundles and HC bodies were measured using a 10×40 eyepiece reticule. Each square of the reticule was 100 μm on a side. Surviving hair bundles and HC bodies were counted in each of three randomly selected fields including both striolar and extrastriolar regions, and the values obtained were averaged. Means and SDs were then converted into percentages for illustration in the figure. Five utricles were examined in each condition (n=5), based on our prior experience with treatment variability.

Effect of notch inhibition. To induce HC-like cells, two days after GM damage, we cultured utricular explants with 20 μM DAPT (N-[N-[(3,5-Difluoropheny)acetyl]-L-alanyl]L-phenylglycinetert-butyl: C₂₃H₂₆F₂N₂O₄, Abcam) for 2 days, followed by 5 days without DAPT. DAPT is a prototypical y-secretase inhibitor. Since Notch signaling requires γ-secretase activity, DAPT is a potent inhibitor of this signaling pathway. As a control, we cultured GM-damaged utricles for 7 days without DAPT. Explants were analyzed for myosin7A immunoreactivity both as whole mounts and as tissue sections

Transduction of Ad-espins. Utricular maculae were cultured in DMEM overnight and then damaged with GM for two days. On the third day, we transduced 5 explants each with Ad-E1 or Ad-E4 (1×10⁷ pfu/ml) overnight. Using the same conditions, we transduced adenovirus GFP (Ad-GFP) as a control. Explants were then cultured in DMEM for 7 days. For Notch inhibition, DAPT (20 μM) was applied for 2 days, in between GM damage and adenovirus transduction.

Observation GFP expression using time-lapse microscopy. Beginning 70 hrs after adenovirus transduction, we evaluated GFP expression using time-lapse microscopy (BZ9000, Keyence, Japan). Each of the five samples was observed three times per hr (20 mins) from 24 hrs after transduction up to 70 hrs. This method was used to identify the time of maximal GFP-adenoviral expression.

Histochemical analysis. At the end of the culture period, explants were fixed for 15 min in 4% paraformaldehyde and permeabilized with 5% Triton X-100 in phosphate-buffered saline (PBS) after 3×PBS wash. Explants were then exposed to a blocking solution containing 10% bovine serum albumin (BSA), and then incubated with anti-myosin7A rabbit polyclonal antibodies (25-6790; Proteus Bioscience Inc., Ramona, Calif., USA; 1:500). Alexa-Fluor 568 goat anti-rabbit IgG (A-11011, Invitrogen, Calif., USA; 1:100) was used as the secondary antibody. At the same time, specimens were incubated in FITC-conjugated or Alexa-Fluor 633 phalloidin (1:100; Invitrogen) to label F-actin for 1 hr. Finally, the explants were incubated with DAPI for 15 min. Specimens were examined using a Leica TCS-SP2 laser-scanning confocal microscope (Leica Microsystems Inc., Wetzlar, Germany). Cells with phalloidin-positive surface structures were counted in randomly selected areas and averaged in each sample for each condition, using an evaluation reticule 100 μm on each side. Results from 6-7 samples were evaluated, based on initial observations of variability.

Observation of stereociliary bundles by scanning electron microscopy (SEM). After the elimination of most stereociliary bundles by GM exposure, the process of hair bundle re-emergence induced by espin transduction was assessed by SEM. The explants were fixed with 4% paraformaldehyde and 0.05% glutaraldehyde at 4° C. for 4 hrs. After fixation, they were dehydrated, dried, and coated with a thin layer of platinum palladium. The specimens were examined with a Hitachi S-4700 scanning electron microscope (Hitachi, Tokyo, Japan). Structures which exhibited clear stereociliary characteristics were counted in standard, randomly selected areas and averaged for each condition (n=6-8 explants, based on the variability exhibited by explants). Each square of the evaluation reticule employed was 30 μm on each side.

Functional analysis with FM1-43FX. To evaluate the physiological function of surviving HCs, we exposed GM-treated, DAPT exposed and transfected explants to FM1-43 (3-[4-[2-[4-(dibutylamino)phenyl]ethenyl] pyridin-1-ium-1-yl]propyl-triethylazanium dibromide: C₃₀H₄₉Br₂N₃) FX dye (5 μM, Invitrogen). FM1-43 is a lipophilic, fluorescent dye that passes very rapidly through the MET channels located on HC stereociliary bundles. For this reason, HC fluorescence after brief exposure to FM1-43 is commonly used to verify the functional status of the cells. The explants were transferred to culture media supplemented with 5 μM FM1-43 for 10 seconds. During FM1-43 incubation, we applied mechanical stimulation via a fluid stream from a pipette, as described previously (36). After fixation with 4% PFA, the specimens were examined with a TCS-SP2 laser-scanning confocal microscope. Three independent assays were performed in each condition. FM1-43 positive cells were counted in randomly selected areas and averaged for each condition (n=6-9, based on prior studies). Each square of the evaluation reticule was 100 μm on each side.

Statistical analysis. Sample sizes were chosen based on our prior experience with variability in GN-exposed macular explants, to detect substantial differences in the measured variables. Samples were excluded from the experiments if they failed to attach to the culture surface or were folded. Because all samples were essentially identical, no randomization was performed. Analysis was unblinded. Statistical analysis was performed by analysis of variance (ANOVA) followed by the least significant difference (LSD) post-hoc test with Bonferroni correction for repeated measures and two-sided t-test (Stat View 5.0), after determination of normal distribution and comparable variances between samples. Differences associated with P values of less than 0.05 were considered to be statistically significant. All data are presented as mean ±standard deviation (SD).

### Results

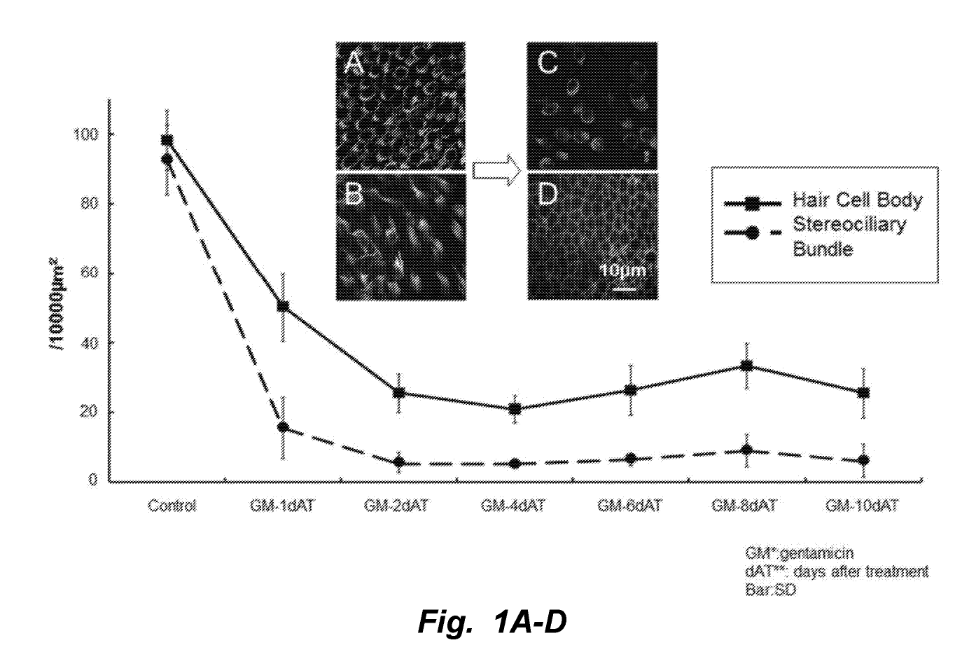

Gentamicin damage to the HC stereociliary bundles and cell bodies. We evaluated the number of HC somata and stereociliary bundles after gentamicin (GM) treatment of utricular maculae in vitro (FIG. 1). After GM treatment, stereocilliary bundles were almost entirely lost from the macular epithelium (FIG. 1D) and the number of HC bodies was dramatically decreased. However, after an initial steep decline in HCs, the number was stable out to 10 days. Thus, a subset of HC somata, absent their stereocilia, remained even 10 days after GM damage (FIG. 1C).

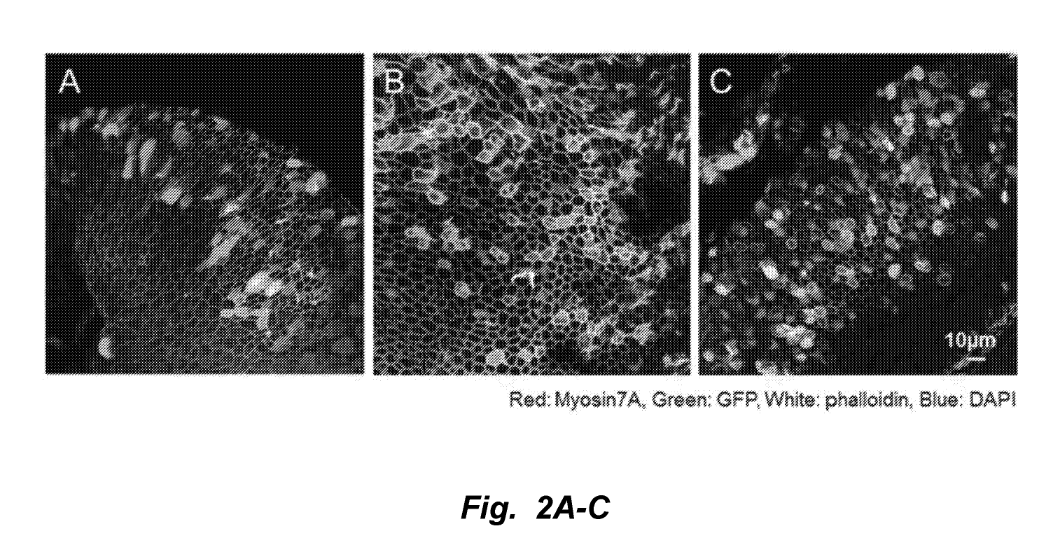

Transduction of macular explants. We examined the transduction of GM-treated macular explants by Adenovirus-GFP (Ad-GFP), Ad.E1-EGFP (Ad-E1) and Ad.E4-EGFP (Ad-E4) adenoviral vectors. The maculae were well transduced by each vector, and the efficiencies of the three transductions were similar. Both HCs and supporting cells were transduced (FIG. 2). The expression of GFP in Ad-GFP transduced cells was diffused throughout the cell bodies (FIG. 2A). In contrast, filamentous patterns of GFP were observed in Ad-E1 and Ad-E4 transduced cells (FIG. 2B, 2C). These expression patterns appeared to be related to the actin-binding characteristics of espin.

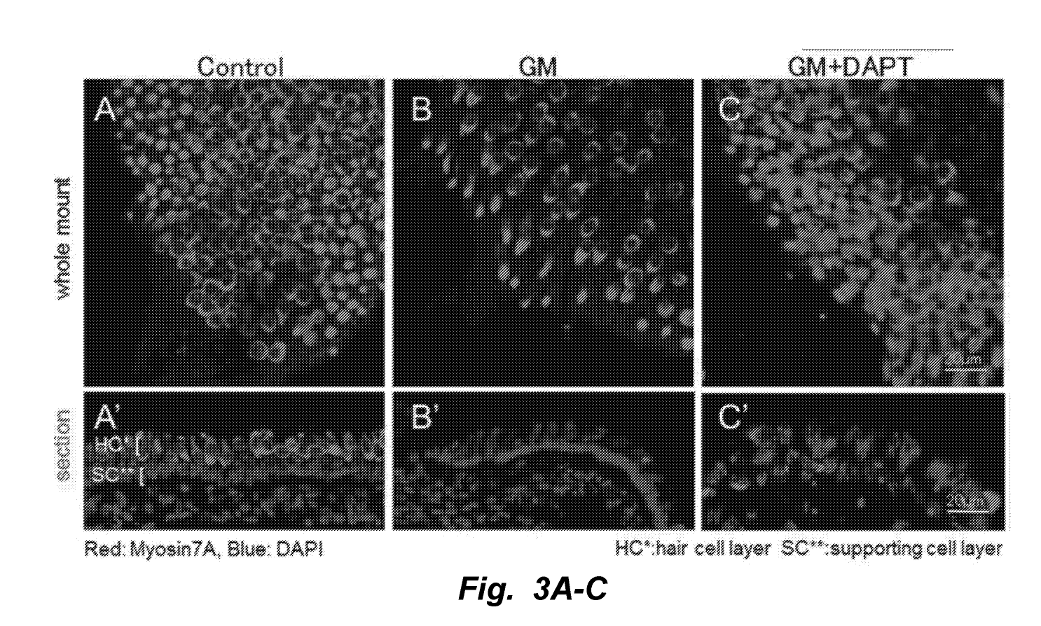

The effect of DAPT treatment. As noted above, after GM damage, the number of HCs present in macular explants decreased dramatically (FIG. 3A, 3A′, 3B,3B′). However, damaged explants treated with DAPT exhibited substantially more cells expressing the HC marker myosin7A, especially at the edges of the explants. In sectioned samples, these myosin7A-positive cells were observed to lie primarily in the supporting cell layer (FIG. 3C, 3C′). The number of myosin7A-positive cells in GM damaged explants and DAPT-treated explants were respectively: 25.4±5.5 (SD) and 38.5±8.64 (SD)/10000 μm2 (P<0.05, t test). However, stereociliary bundles remained very sparse in the DAPT-treated explants.

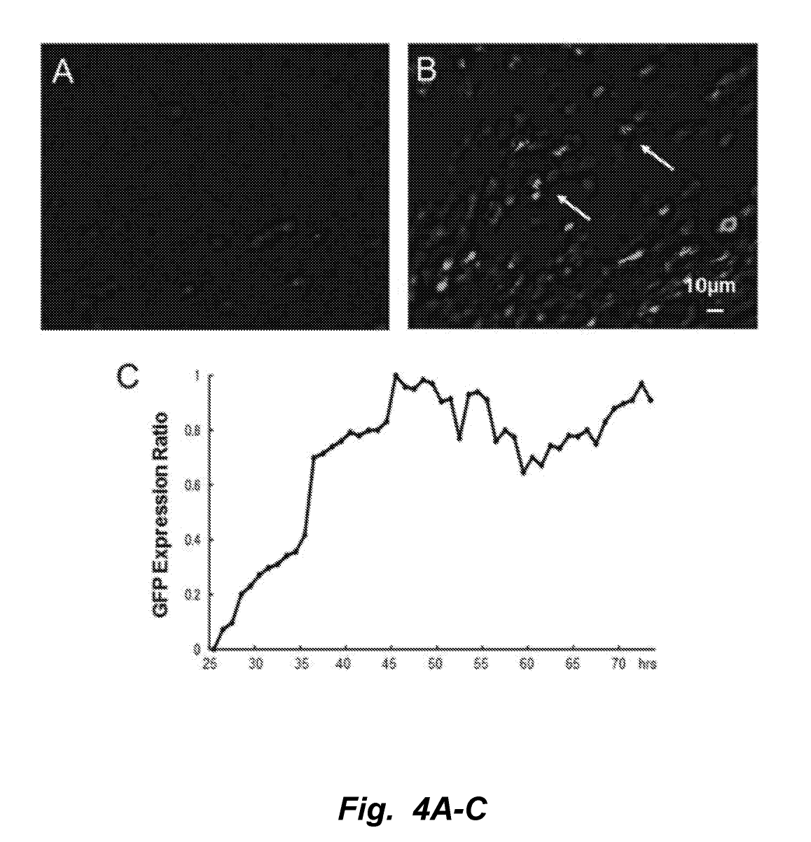

Time-lapse microscopy after adenoviral transduction. Espinl-GFP expression after Ad-E1 transduction of GM-damaged, DAPT-treated explants was examined by time-lapse microscopy. Twenty-four hrs after vector administration, almost no GFP-positive cells were observed (FIG. 4A). However, by 31 hrs, GFP expression had increased. By 46-48 hrs, expression reached a visual maximum and extensive filamentous GFP expression was observed (FIG. 4B, 4C, arrows). GFP expression then declined, but remained apparent for an additional 24 hrs. GFP signals following transduction with the other 2 adenoviruses (Ad-GFP and Ad-E4) increased, peaked, and declined at a slightly earlier stage.

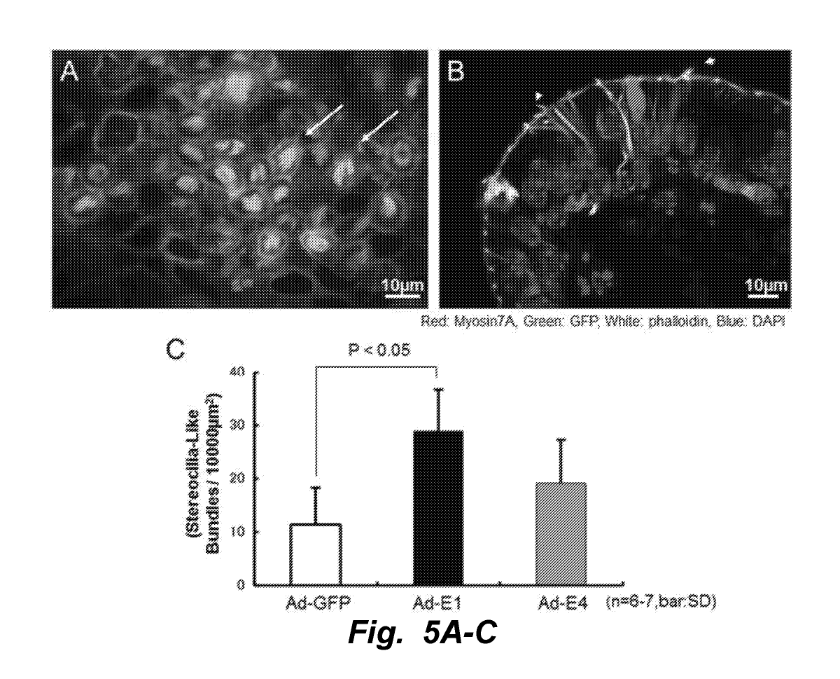

Influence of espin gene transduction on stereociliary bundles. Transduction of damaged and regenerated explants with Ad-GFP or Ad-E4 had no effect on the number of stereociliary bundles. However, on the surfaces of Ad-E1 transduced explants a large increase in the number of phalloidin-positive structures which resembled immature stereociliary bundles was noted (FIG. 5A). In sectioned explants, the many myosin7A-positive cells that were apparent in the supporting cell layer exhibited GFP-positive extensions that reached from the supporting cell layer to the apical surface of the epithelium. Moreover, phalloidin-labeled stereociliary bundle-like structures projected from the apical surfaces of these extensions (FIG. 5B). While some stereociliary bundles were observed in Ad-GFP- or Ad-E4-transduced explants (11.4±6.87 (SD)/10000 μm²), a substantially and significantly greater number were observed on Ad-E1-transduced macular explants (28.8±7.82 (SD)/10000 μm²; .P<0.05, t-test) (FIG. 5C).

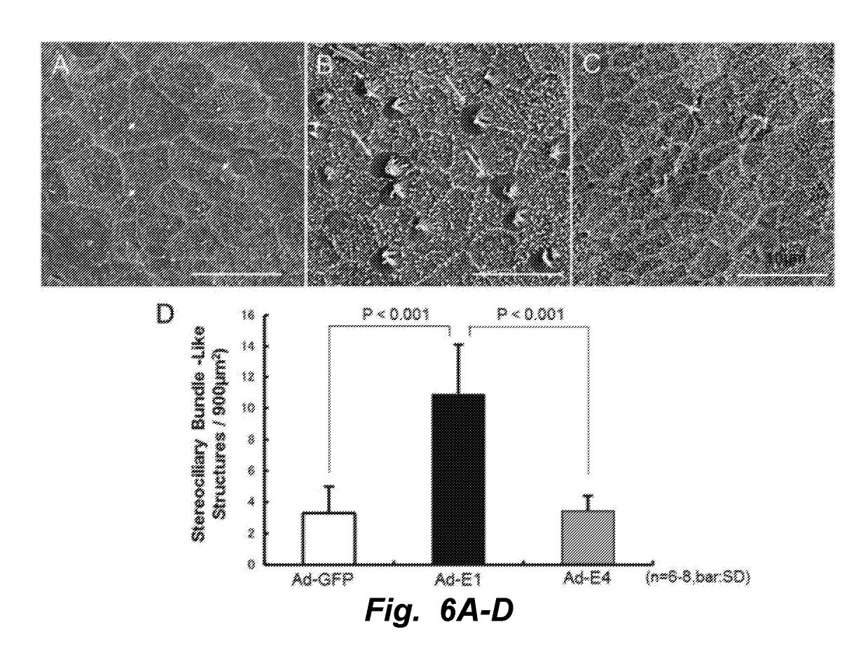

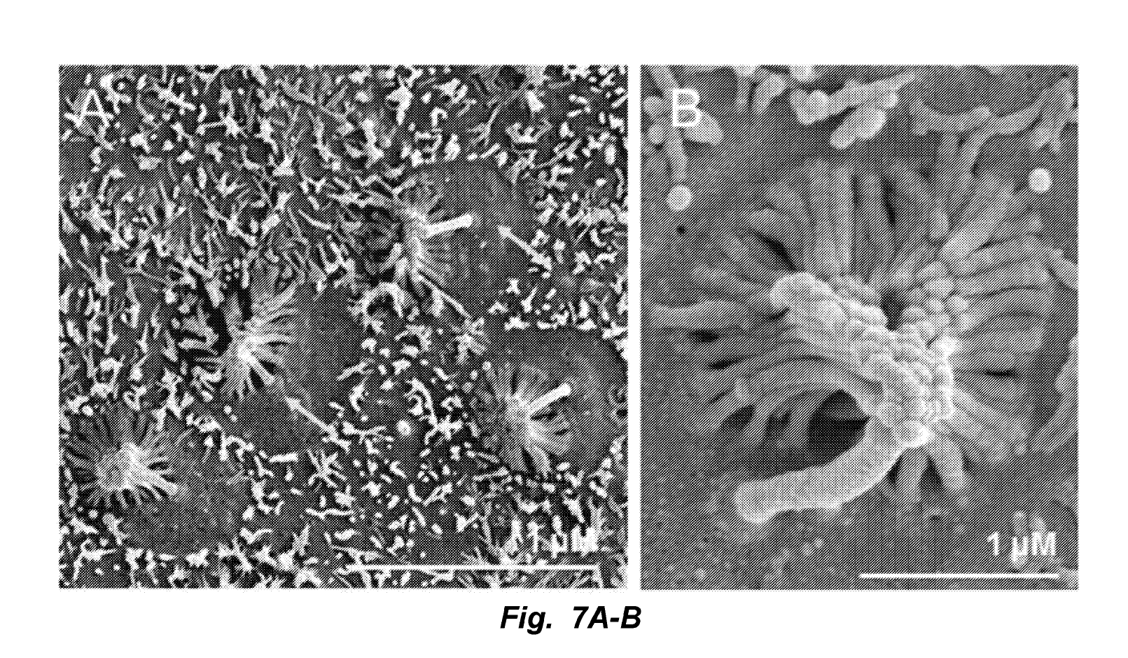

Scanning electron microscopy (SEM). After Ad-GFP transduction, the reticulated boundaries between cells were clearly apparent on the surfaces of explants imaged by SEM. Small microvilli and basal bodies were also observed on the surfaces of the cells, but stereociliary bundles were essentially absent (FIG. 6A). In contrast, on the surface of Ad-E1 transduced explants, many immature stereociliary bundle-like structures were observed (FIG. 6B). However, there were very few immature stereociliary bundle-like structures in Ad-E4 transduced explants, although elongated microvillus-like elements were seen (FIG. 6C). When the stereociliary bundle-like structures were quantified, the number on Ad-E1 transduced explants was substantially and significantly (p<0.05, t-test) greater than in Ad-GFP or Ad-E4 transduced epithelia (FIG. 6D). Higher magnification images revealed recognizable bundles of stereocilia, with a typical stair-step arrangement, on Ad-E1 transduced explants. These bundles exhibited a single, central kinocilium, and stereocilia were present on approximately one half of the apical surface of each bundle-bearing cell (FIG. 7A). These stereocilia appeared to be linked at their tops (FIG. 7B).

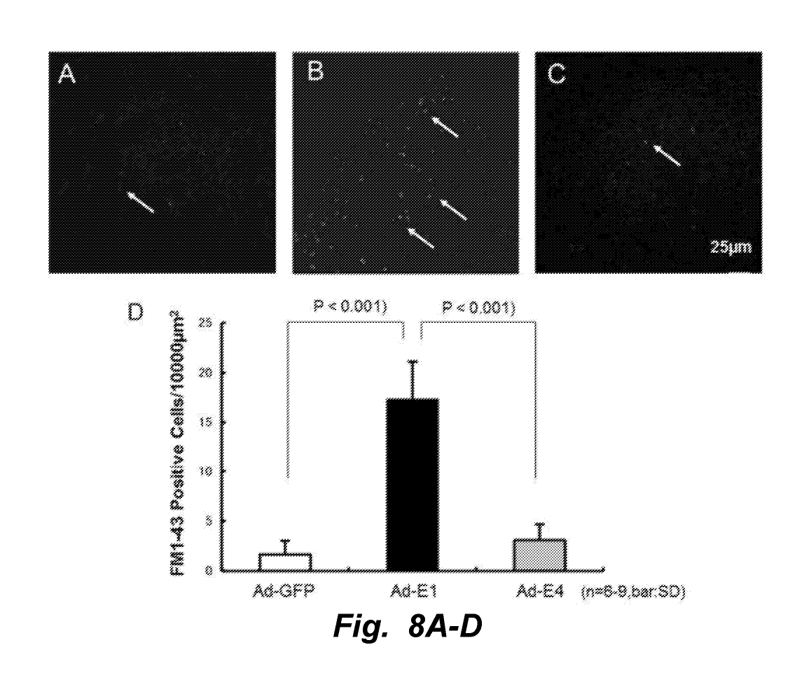

Functional analysis of stereociliary bundles using FM1-43FX loading. After brief treatment with FM1-43FX, only a small number of fluorescent cells were observed in Ad-GFP and Ad-E4 transduced explants (FIG. 8A, 8C). However, substantially more FM1-43FX-positive cells were observed in Ad-E1 transduced explants (FIG. 8B). Quantitative analysis of FM1-43 positive cells in explants revealed dramatic and significant (p<0.05) differences in labeling between Ad-E1 transduced explant and those transduced with either Ad-GFP or Ad-E4 (FIG. 8D). These results provide strong evidence that stereociliary bundles on Ad-E1 transduced explants exhibit functional mechanoelectrical transduction channels.

### Discussion

Gene therapy has proven to be a promising tool for the correction of gene deficiencies and for introducing beneficial gene expression into a variety of tissues. Recently, advances have been reported in the use of gene therapy for inner ear disorders (12). In this study, we evaluated the potential for gene therapy-induced espin overexpression to regenerate HC stereociliary bundles after HC damage and induction. When HCs were damaged with aminoglycosides, their apical surface including the hair bundle was rapidly lost, as has been previously reported (4). This was followed by the degeneration of the HC soma for most cells. However, a subpopulation of HCs survived, absent their stereocilia, even at 10 days post aminoglycoside treatment. Again, this has been reported in prior studies (19). Treatment of damaged cultures with the y-secretase inhibitor DAPT resulted in the generation of additional HCs, but these cells lacked stereociliary bundles in our study. This agrees with the results of some prior evaluations of HC regeneration (17,19) although stereocilia have been reported on regenerated mammalian HCs in other studies, especially when longer periods of regeneration are involved (15, 18) Transduction with Espinl-EGFP in an adenoviral vector led to robust growth of stereociliary bundles, while transduction with an Espin4-EGFP or GFP alone was ineffective.

As noted above, espins are actin bundling proteins produced in multiple isoforms from a single gene. Espin mutant mice show abnormal stereocilia, indicating the importance of this gene for these structures (9). Moreover, specific espin isoforms have the potential to be differentially involved in stereociliogenesis during HC development. In the inner ear HCs of altricial rodents, the espin1 isoform accumulates during the late embryonic and early postnatal periods, while the espin2 and 3 isoforms predominate in the embryonic inner ear until approximately E20. Therefore, espins1-3 are candidates for involvement in stereociliagenesis, with espin1 expression most closely matching the period of stereociliary development (9). In contrast, the espin4 isoform accumulates between postnatal days 6 and 10, after stereocilia have reached their essentially adult characteristics (9). This suggests that espin4 is less likely to be involved in stereociliagenesis. However, when espin4 is over-expressed in other cell types, microvilli are elongated (23) and in cochlea both microvilli and stereocilia are similarly elongated (24). Therefore, more than one espin isoform has the capacity to influence stereocilia formation. We therefore tested the ability of espin1 and espin4 overexpression to generate stereocilia in damaged and regenerated HCs. In Ad-E1 transduced explants, we observed phalloidin-positive stereocilia bundle-like structures on myosin7A-positive cells. SEM study revealed that only in Ad-E1 transduced explants did we observe recognizable stereocilia with a staircase pattern. This, in combination with the data cited above, suggests that espin4 may be more involved in the elongation than in the genesis of stereocilia. However, it should of course be noted that the fusion of espin4 to GFP might have altered its properties in our experiments. Any conclusions regarding the lack of effect of espin4 transduction on stereociliary bundle formation should keep this in mind.

These results suggest that espin1 gene therapy might be effective as a means of regenerating stereociliary bundles. However, it does not necessarily follow that the stereocilia are functional. Rapid FM1-43 accumulation in HCs, on the order of seconds, is thought to be mediated by entry through functional mechanoelectrical transduction channels (25,26). Although endocytotic entry has been reported in guinea pig inner HCs, this occurs with substantially slower kinetics (27). Therefore, rapid FM1-43 loading is thought to reflect the physiological function of HC mechanotransduction channels at the tips of the stereocilia (26). In Ad-E1 transduced explants, the number of FM1-43 positive cells observed after brief exposure was substantially and significantly (p<0.05, t-test) larger than the other two experimental groups. This suggests that stereocilia induced by espin1 gene therapy bear functional MET (mechanoelectrical transduction) channels.

The fact that espin1 overexpression alone increased the formation of stereociliary arrays is perhaps surprising, given that actin-crosslinking presumably represents only part of the process required for stereociliary formation. This suggests that the genetic program for stereociliagenesis is partially active in damaged and/or regenerated HCs, but lacks espn gene expression as a critical component. The reason for this lack is unclear. It can be assumed that espin expression during development is initiated by the activity of specific HC fate and differentiation genes. Transduction of nonsensory inner ear cells with the transcription factor ATOH1 induces the formation of HC-like cells, some of which will form stereocilia, although they are often immature (13,18). ATOH1 overexpression has also been shown to induce stereociliagenesis in damaged HCs, although again some such stereocilia are immature (28). Therefore ATOH1 alone does not appear to be sufficient to specify appropriate espn gene expression. ATOH1 is known to directly regulate the gene encoding the HC differentiation transcription factor POU4F3 (29), and animals that lack the pou4f3 gene fail to develop stereocilia (30). It is thus possible that POU4F3 directly regulates the espn gene. We therefore evaluated the 1500 bp region 5′ to the start site of the murine espn gene for consensus POU4F3 binding sites, as identified by Xiang et al., (31) or the JASPAR database. Two sites were found ˜650-800 bp 5′ to the espn transcription initiation site. This provides suggestive, but by no means sufficient, evidence of POU4F3 regulation. In any case, low levels of expression of POU4F3, or of other HC differentiation factors, may explain the insufficient espn transcription in damaged and regenerated HCs.

Differentiated HCs express Notch, and the Notch pathway inhibits adjacent supporting cells from differentiating into HCs (32-34). Lin et al. (19) reported that HC regeneration was promoted by Notch inhibition, with conversion of supporting cells to HCs and increased atoh1 transcriptional activity. This is consistent with our results in which many more Myosin7A-positive cells were observed in damaged epithelia that were treated with DAPT. In our study, we did not observe stereociliary bundles on the myosin7A-positive cells induced by Notch inhibition alone within 7days. This suggests that Notch signaling may not be sufficient for complete development of HCs, which might otherwise be expected if Notch effects are mediated by increased ATOH1. Alternatively, stereocilliary regeneration induced by Notch inhibition may require additional time.

In conclusion, espin1 gene therapy appears be useful for significantly enhancing morphological and physiological stereociliary bundle regeneration on damaged and regenerated HCs. The combination of Notch inhibition and espin1 gene transduction is therefore a promising candidate for inner ear regenerative therapy in future. However, it must be noted that our experiments were performed in neonatal mice. Future studies will be needed to determine whether this regenerative effect occurs in adult mice, a necessary pre-requisite for potential clinical utility.

### REFERENCES

1. Nadol Jr J B. Hearing loss. N Engl J Med 1993; 329: 1092-1102.

2. Roberson D F, Weisleder P, Bohrer P S, Rubel E W. Ongoing production of sensory cells in the vestibular epithelium of the chick. Hear Res 1992; 57: 166-174.

3. Ryals B M, Rubel E W. Hair cell regeneration after acoustic trauma in adult Coturnix quail. Science. 1988; 240: 1774-1776.

4. Gale J E, Meyers J R, Periasamy A, Corwin J T. Survival of bundleless hair cells and subsequent bundle replacement in the bullfrog's saccule. J Neurobiol. 2002; 50: 81-92.

5. Jia S, Yang S, Guo W, He D Z. Fate of mammalian cochlear hair cells and stereocilia after loss of the stereocilia. J Neurosci. 2009; 29: 15277-15285.

6. Sekerkova G, Zheng L, Loomis P A, Mugnaini E, Bartles J R. Espins and the actin cytoskeleton of hair cell stereocilia and sensory cell microvilli. Cell Mol Life Sci. 2006; 63: 2329-2341.

7. Sekerkov G, Zheng L, Mugnaini E, Bartles J R. Differential expression of espin isoforms during epithelial morphogenesis, stereociliogenesis and postnatal maturation in the developing inner ear. Dev. Biol. 2006; 291: 83-95.

8. Li H, Liu H, Balt S, Mann S, Corrales C E, Heller S. Correlation of expression of the actin filament-bundling protein espin with stereociliary bundle formation in the developing inner ear. J. Comp. Neurol. 2004; 468: 125-134.

9. Rzadzinska A, Schneider M, Noben-Trauth K, Bartles J R, Kachar B. Balanced levels of Espin are critical for stereociliary growth and length maintenance. Cell Motil Cytoskeleton. 2005; 62: 157-165.

10. Zheng L, Sekerkova G, Vranich K, Tilney L G, Mugnaini E, Bartles J R. The deaf jerker mouse has a mutation in the gene encoding the espin actin-bundling proteins of hair cell stereocilia and lacks espins. Cell. 2000; 102: 377-385.

11. Van de Water T R, Staecker H, Halterman M W, Federoff H J. Gene therapy in the inner ear. Mechanisms and clinical implications. Ann N Y Acad Sci. 1999; 884: 345-360.

12. Ryan A F, Dazert S. Gene therapy for the inner ear: challenges and promises. Adv Otorhinolaryngol. 2009; 66: 1-12

13. Kawamoto K, Yagi M, Stover T, Kanzaki S, Raphael Y. Hearing and hair cells are protected by adenoviral gene therapy with TGF-beta 1 and GDNF. Mol. Ther. 2003; 7: 484-492.

14. Kawamoto K, Sha S H, Minoda R, Izumikawa M, Kuriyama H, Schacht J et al. Antioxidant gene therapy can protect hearing and hair cells from ototoxicity. Mol Ther 2004; 9: 173-181.

15. Izumikawa M, Minoda R, Kawamoto K, Abrashkin K A, Swiderski D L, Dolan D F et al. Auditory hair cell replacement and hearing improvement by Atoh1 gene therapy in deaf mammals. Nat Med 2005; 11: 271-276.

16. Schlecker C, Praetorius M, Brough D E, Presler R G Jr, Hsu C, Plinkert P K, et al. Selective atonal gene delivery improves balance function in a mouse model of vestibular disease. Gene Ther. 2011; 18: 884-890.

17. Tona Y, Hamaguchi K, Ishikawa M, Miyoshi T, Yamamoto N, Yamahara K, et al. Therapeutic potential of a gamma-secretase inhibitor for hearing restoration in a guinea pig model with noise-induced hearing loss. BMC Neurosci. 2014; 15: 66.

18. Mizutari K, Fujioka M, Hosoya M, Bramhall N, Okano H J, Okano H, et al. Notch inhibition induces cochlear hair cell regeneration and recovery of hearing after acoustic trauma. Neuron. 2013; 77: 58-69.

19. Lin V, Golub J S, Nguyen T B, Hume C R, Oesterle E C, Stone J S. Inhibition of Notch activity promotes nonmitotic regeneration of hair cells in the adult mouse utricles. J Neurosci. 2011; 31: 15329-39.

20. Ahmed M, Xu J, Xu P X. EYA1 and SIX1 drive the neuronal developmental program in cooperation with the SWI/SNF chromatin-remodeling complex and SOX2 in the mammalian inner ear. Development. 2012; 139: 1965-77.

21. Atkinson P J, Huarcaya Najarro E, Sayyid Z N, Cheng A G. Sensory hair cell development and regeneration: similarities and differences. Development. 2015; 142: 1561-1571.

22. Ryan A F. The cell cycle and the development and regeneration of hair cells. Curr Topics Devel Biol 2002; 57: 449-466.

23. Loomis P A, Zheng L, Sekerkova G, Changyaleket B, Mugnaini E, Bartles J R. Espin cross-links cause the elongation of microvillus-type parallel actin bundles in vivo. J Cell Biol. 2003; 163: 1045-1055.

24. Rzadzinska A, Schneider M, Noben-Trauth K, Bartles J R, Kachar B. Balanced levels of Espin are critical for stereociliary growth and length maintenance. Cell Motil Cytoskeleton. 2005; 62: 157-165.

25. Gale J E, Marcotti W, Kennedy H J, Kros C J, Richardson G P. FM1-43 dye behaves as a permeant blocker of the hair-cell mechanotransducer channel. J Neurosci. 2001; 21: 7013-7025.

26. Meyers J R, MacDonald R B, Duggan A, Lenzi D, Standaert D G, Corwin J T, et al. Lighting up the senses: FM1-43 loading of sensory cells through nonselective ion channels. J Neurosci. 2003; 23: 4054-4065.

27. Griesinger C B, Richards C D, Ashmore J F. FM1-43 reveals membrane recycling in adult inner hair cells of the mammalian cochlea. J Neurosci. 2002; 22: 3939-3952.

28. Yang S M, Chen W, Guo W W, Jia S, Sun J H, Liu H Z, et al. Regeneration of stereocilia of hair cells by forced Atoh1 expression in the adult mammalian cochlea. PLoS One. 2012; 7: e46355.

29. Masuda M, Dulon D, Pak K, Mullen L M, Li Y, Erkman L, et al. Regulation of POU4F3 gene expression in hair cells by 5′ DNA in mice. Neuroscience. 2011; 197: 48-64.

30. Erkman L, McEvilly R J, Luo L, Ryan A K, Hooshmand F, O'Connell S M, et al. Role of transcription factors Brn-3.1 and Brn-3.2 in auditory and visual system development. Nature. 1996; 381: 603-606, 1996.

31. Xiang M, Zhou L, Macke J P, Yoshioka T, Hendry S H, Eddy R L, et al. The Brn-3 family of POU-domain factors: primary structure, binding specificity, and expression in subsets of retinal ganglion cells and somatosensory neurons. J Neurosci. 1995; 15: 4762-4785.

32. Yamamoto N, Tanigaki K, Tsuji M, Yabe D, Ito J, Honjo T. Inhibition of Notch/RBP-J signaling induces hair cell formation in neonate mouse cochleas. J Mol Med (Berl) 2006; 84: 37-45.

33. Hori R, Nakagawa T, Sakamoto T, Matsuoka Y, Takebayashi S, Ito J. Pharmacological inhibition of Notch signaling in the mature guinea pig cochlea. Neuroreport 2007; 18: 1911-1914.

34. Murata J, Ikeda K, Okano H. Notch signaling and the developing inner ear. Adv Exp Med Biol. 2012; 727: 161-173.

35. He T C, Zhou S, da Costa L T, Yu J, Kinzler K W, Vogelstein B. A simplified system for generating recombinant adenoviruses. Proc Natl Acad Sci U S A. 1998; 95: 2509-2514.

36. Taura A, Kojima K, Ito J, Ohmori H. Recovery of hair cell function after damage induced by gentamicin in organ culture of rat vestibular maculae. Brain Res. 2006; 1098: 33-48.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

| INFORMAL SEQUENCE LISTING | |
|---|---|
| human espin isoform 1 nucleic acid sequence (mRNA; NCBI | |
| Reference Sequence: NM_031475.2) | |
| Sequence ID No: 1 | |
| 1 | agcggagcgc caggcagcgc ggagcggagg ccaggcccac agccgctccg cctcccggcc |
| 61 | cgcagatccc cgacggccgc accgcgggct cctctggccc gcaagaacac gtgcatggcg |
| 121 | tcctggggaa ggcgctgagt gcggagtcgc ggcgccgcac gcggcaccat ggccctggag |
| 181 | caggcgctgc aggcggcgcg gcagggcgag ctggacgtgc tgaggtcgct gcacgccgca |
| 241 | ggcctcctgg ggccctcgct gcgcgacccg ctggacgcgc tgcccgtgca ccacgcggcc |
| 301 | cgcgctggga agctgcactg tctgcgcttc ctggtggagg aagccgccct ccccgccgcg |
| 361 | gcccgcgccc gcaacggcgc cacaccggcc cacgacgcct ccgccaccgg ccacctcgcc |
| 421 | tgcctgcagt ggctgctgtc gcagggcggc tgcagagtgc aggacaaaga caattctggt |
| 481 | gccacagtct tgcatctggc tgcccgcttc ggccaccccg aggtggtgaa ctggctcttg |
| 541 | catcatggcg gtggggaccc caccgcggcc acagacatgg gcgccctgcc tatccactac |
| 601 | gctgccgcca aaggagactt cccctccctg aggcttctcg tcgagcacta ccctgaggga |
| 661 | gtgaatgccc aaaccaagaa cggtgccacg cccctgtacc tggcgtgcca ggagggccac |
| 721 | ctggaggtga cccagtacct ggtgcaggaa tgcggcgcag acccgcacgc gcgcgcccac |
| 781 | gacggcatga ccccgctgca cgccgcggcg cagatgggcc acagcccagt catcgtgtgg |
| 841 | ttggtgagct gcaccgacgt gagcctgtcc gagcaggaca aagacggcgc caccgccatg |
| 901 | cacttcgcgg cgagccgcgg ccacaccaag gtgctcagct ggctgctgct gcacggcggg |
| 961 | gagatctcgg ctgacctgtg gggcgggacc ccgctgcacg cgccgccga gaacggggag |
| 1021 | ctagagtgct gccagatcct ggtagtgaac ggcgcggagc tggacgtccg cgaccgcgac |
| 1081 | gggtacacgg ccgccgacct gtcggacttc aacggccaca gccactgcac ccgctacctg |
| 1141 | cgcacggtgg agaacctgag cgtggagcac cgcgtgcttt cccgggatcc atccgcagag |
| 1201 | ctggaggcta agcagccgga ttcaggcatg tcctcaccca ataccacggt gtcggtccag |
| 1261 | ccgctgaact ttgacctcag ctcgcctacc agcaccctct ccaactacga ctcctgctcc |
| 1321 | tccagccact ccagcatcaa gggccagcac cctccatgtg ggctttccag cgctagagct |
| 1381 | gcagacatac agagctacat ggacatgctg aacccggagc tgggcctgcc tcggggcacg |
| 1441 | attgggaagc ccacaccccc accaccccca cccagcttcc ccccgccacc cccgccccca |
| 1501 | ggcacccaac tgcccccacc cccacctggc tacccagctc ccaagcctcc tgtaggacca |
| 1561 | caggcagctg acatctacat gcagaccaag aacaaactcc gccacgtgga gacagaggcc |
| 1621 | ctcaagaagg agctgagctc ctgtgacggc cacgacgggc tgcggaggca ggactccagc |
| 1681 | cgcaagcccc gcgccttcag caagcagccc agcacggggg actactaccg gcagctgggc |
| 1741 | cgctgccccg gcgagacgct ggccgcacgc ccgggcatgg cgcacagcga ggaggtgcgt |
| 1801 | gcccgccagc ccgcgcgcgc cggctgcccg cgcctcggcc ctgccgcccg cggctcactc |
| 1861 | gaaggcccct ccgctccccc gcaggcggcg ctgcttcctg ggaaccatgt tcctaacggc |
| 1921 | tgcgccgcgg accccaaggc gtccagggag ctgccaccgc cgcccccacc gccgccgccg |
| 1981 | cccctgccgg aggccgcgag ttcgccaccg ccggccccgc ctctgcccct cgagagcgct |
| 2041 | ggccctggct gcgggcagcg ccgctcctcc tcgtccaccg gcagcaccaa gtctttcaac |
| 2101 | atgatgtccc cgacgggcga caactcggag ctactggctg agattaaggc aggcaagagc |
| 2161 | ctgaagccga cgccccagag caaggggctg accacagtgt tctcaggcat cgggcagccg |
| 2221 | gccttccagc ccgattcgcc gctgccttct gtgtcacctg cactgtcacc agtccggagc |
| 2281 | cccacaccgc cagctgcggg gtttcagccg ctgctcaatg gaagcttggt tcccgtgccg |
| 2341 | cccactactc ctgcgccggg agtgcagctg gacgtggagg ctctcatccc cacgcacgat |
| 2401 | gagcagggcc ggcccatccc cgagtggaag cgccaggtga tggtgcgcaa gatgcagctg |
| 2461 | aagatgcagg aggaggagga gcagaggcgg aaggaggagg aggaggaggc ccggctggcc |
| 2521 | agcatgcccg cctggaggcg ggacctcctg cggaagaagc tggaagaaga gagggagcag |
| 2581 | aagcggaaag aggaggagcg acagaagcag gaggagctgc ggcgggagaa ggaacagtca |
| 2641 | gagaagctgc ggacgctggg ctacgatgag agcaagctgg cgccctggca gcgacaggtc |
| 2701 | atcctgaaga agggggacat cgctaagtac tagaggccgc agactcctgt ccgcagcctc |
| 2761 | gcagctccgt ggggccctcc gccccagccc cagccagcca ggccctggtg gaaaggctgg |
| 2821 | gagccgcaca gccctcccct cctgcgctgg aaaccctccc tgacccccac cctggccccc |
| 2881 | cgtatcccca gcccttggca acactggagt gcacacgccg ccacggttgc ccagaaaaag |
| 2941 | tgcccaagct gctgacgcaa acaacaacaa atgctgctta tttgcatgcc gacttacata |
| 3001 | tatttgcatg ttcgttgact atcaaagagt gcagagctct ccccagcccc gtgggtggtg |
| 3061 | actttgtttt cctgcggggc tcagccccct ccaggatgca gccccctccc ccgcaccccg |
| 3121 | gaaccggcgt cgctggcgca tcctgggtgg aggcaggccc cgagctcggg gaaggggttt |
| 3181 | tcccttcctc tctgacccag atctgcgcgc ggcctagccc gggcctcatt tcttatcccc |
| 3241 | gccaagggtt tcctctcagt catttgttta ccagaaacat gaaaactgcc tgtctggccg |
| 3301 | ggccgcactt gtggcccccg ggaccccacc tctggcccca cctccctcaa gtctgcgccc |
| 3361 | cgtccccagc cagacccact cgctgccggg accctttcac tgccccggtg gagtgaatag |
| 3421 | aggatgaggg gccctgaccc tgtgtctcca actgctgcac cccatcccga ccctgtctcc |
| 3481 | gccacctcgc agccccatta aagcgctctc atctgggctc cggttcactc a |
| human espin isoform 1 amino acid sequence (NCBI Reference | |
| Sequence: NP_113663.2) | |
| Sequence ID No: 2 | |
| 1 | maleqalqaa rqgeldvlrs lhaagllgps lrdpldalpv hhaaragklh clrflveeaa |
| 61 | lpaaararng atpandasat ghlaclqwll sqggcrvqdk dnsgatvlhl aarfghpevv |
| 121 | nwllhhgggd ptaatdmgal pihyaaakgd fpslrllveh ypegvnaqtk ngatplylac |
| 181 | qeghlevtqy lvqecgadph arandgmtpl haaaqmghsp vivwlvsctd vslseqdkdg |
| 241 | atamhfaasr ghtkvlswll lhggeisadl wggtplhdaa engeleccqi lvvngaeldv |
| 301 | rdrdgytaad lsdfnghshc trylrtvenl svehrvlsrd psaeleakqp dsgmsspntt |
| 361 | vsvqplnfdl ssptstlsny dscssshssi kgqhppcgls saraadiqsy mdmlnpelgl |
| 421 | prgtigkptp pppppsfppp ppppgtqlpp pppgypapkp pvgpqaadiy mqtknklrhv |
| 481 | etealkkels scdghdglrr qdssrkpraf skqpstgdyy rqlgrcpget laarpgmahs |
| 541 | eevrarqpar agcprlgpaa rgslegpsap pqaallpgnh vpngcaadpk asrelppppp |
| 601 | ppppplpeaa sspppapplp lesagpgcgq rrsssstgst ksfnmmsptg dnsellaeik |
| 661 | agkslkptpq skglttvfsg igqpafqpds plpsvspals pvrsptppaa gfqpllngsl |
| 721 | vpvppttpap gvqldveali pthdeqgrpi pewkrqvmvr kmqlkmqeee eqrrkeeeee |
| 781 | arlasmpawr rdllrkklee ereqkrkeee rqkqeelrre keqseklrtl gydesklapw |
| 841 | qrqvilkkgd iaky |
| Mus musculus espin (Espn), transcript variant 1, mRNA (NCBI | |
| Reference Sequence: NM_207687.2) | |
| Sequence ID No: 3 | |
| 1 | atggccctgg agcaggcgct gcaggcggca cggcggggcg acctggacgt gctgaggtcc |
| 61 | ctgcacgccg ccggcctgct ggggccttct ctgcgcgact cgctagacgc cctgccggtg |
| 121 | caccatgcgg cccgctcagg caagctgcac tgtctgcgct acttggtgga ggaggttgcc |
| 181 | ctcccagccg tgtcccgcgc gcgcaacggc gccacaccag cccatgacgc cgccgccaca |
| 241 | ggctacctct cttgcctgca gtggctgctc acacagggtg gctgcagggt gcaggaaaaa |
| 301 | gataactctg gtgccacagt cctgcatctg gctgcccgct ttggccaccc ggatgtggtg |
| 361 | aagtggctgc tgtaccaggg cggtgcaaat tcggccatca ccacagacac gggcgccctg |
| 421 | cctatccact acgctgccgc caaaggagac ctcccctcgc tgaagcttct ggtcgggcat |
| 481 | taccctgagg gagtgaatgc ccaaaccaac aacggtgcca cgcccctgta cctggcgtgc |
| 541 | caggagggcc acctggaagt gacgaagtac cttgtgcagg agtgcagtgc agatccgcac |
| 601 | ctgcgcgccc aagacggcat gacaccccta catgccgcag cacagatggg ccacaaccca |
| 661 | gtcttggtgt ggctggtgag ctttgcagac gttagctttt cagagcagga ccacgacggc |
| 721 | gccacggcca tgcactttgc tgccagtcgc ggccacacca aagtgctcag ctggctcctg |
| 781 | ctgcacgggg cagagatctc gcaggacctg tggggcggaa ccccgctgca tgacgctgct |
| 841 | gagaacgggg aactggagtg ctgccagatc ctcgcggtga acggcgctgg gctggacgtc |
| 901 | cgcgaccacg atgggtacac cgctgccgac ctggcagagt tcaatggcca cacccactgt |
| 961 | tcccgctacc tacgtacggt gcaaaccctg agcttggaac accgagtctt gtcccgggat |
| 1021 | caatccatgg acctggaggc aaagcagctg gactcgggta tgtcctcgcc caacaccacc |
| 1081 | atgtcggtcc agccaatgac ctttgacctg ggctcgccta ctagcacgtt ctccaactat |
| 1141 | gactcctgct cctccagtca ctccagcagc aaggggcagc gatcgaatcg agggattcca |
| 1201 | ggtgcaagag ctgcagactt acagagctac atggacatgc tgaacccaga gaagagcttg |
| 1261 | cctcggggca agctagggaa gccttccccg ccaccacctc caccaccacc accaccaagc |
| 1321 | ttcccgccac ccccaccacc cacaggcacc cagccgcccc cacctccacc aggctaccca |
| 1381 | gctcccaatc cccctgtggg actgcatctg aataacattt acatgcagac caagaacaag |
| 1441 | cttcgccatg tggaggtgga ctcgctcaag gagcccaagg tggagctgaa cgatcagttt |
| 1501 | gcacagccga gctcgggcga cggccactcg gggctacaca ggcaggactc cgggctgctc |
| 1561 | aggcaggatt cggagctgct gcacaggcag gagctgctca ggcacagcac cggactgcgc |
| 1621 | aggcaggact ccgaccgcaa acagcgctcg ttcagtaaac agcccagcac gggggactac |
| 1681 | taccgccagc tgggccgcag cccgggggag ccgctggccg cacgcccggg catggcccac |
| 1741 | agcgaggagg cggcgctgct ccccgggaac cacgtgcaca acggctgctc agcggactcc |
| 1801 | aaagcgtcca gggagctgcc gccgccaccg ccgccgccgc cgctgcccga ggccctgagt |
| 1861 | tcgccgccgc ccgccccacc tctgcccatc gagggcgcgg gcgcagcctg cgggcagcgt |
| 1921 | cgttcctcgt cttctactgg caaagtgaga gtcctgagac acaggaagag caccaaatct |
| 1981 | ttcaacatga tgtccccaac gggtgataac tcagagcttc tggctgagat aaaggcgggc |
| 2041 | aagagcctga agccgacacc gcagagcaag gggctgacaa ccgtgttctc aggcagtggg |
| 2101 | cagccagcct cccagcctga gtcaccgcag cctctggtgt cacctgcgcc atctcggact |
| 2161 | cggagcccca ccccgccagc ctctgggtct cagccactgc tcaatggcag tgtggtgccg |
| 2221 | gcaccacctg ccaccccggc acctggagtc catctggatg tggaggccct cattcccact |
| 2281 | cttgatgagc agggccggcc catcccggag tggaagcgcc aggtgatggt ccgcaagctg |
| 2341 | cagcagaaga tgcaggagga agaggagcag cggaggaagg aggaagagga ggaggcccgg |
| 2401 | ctcgccagcc tgcctgcctg gagacgagac attcttcgga agaagctgga ggaggagagg |
| 2461 | gagcagaagc gaaaagagga ggagcggcaa aagctggagg aaatacagag ggcgaaagaa |
| 2521 | cagtcggaga agctgcggac actaggctac gacgaagcca agctcgcgcc ctggcagcga |
| 2581 | caggtcatct tgaagaaggg ggagatccct aagtaatagg agtctctcgg cttcttgcgt |
| 2641 | gtagcctcac aagttctgag agatggggag cggcccccag cccccgcccc atcctgtcag |
| 2701 | gttggtgcgg aagggctagg atccttgccg cccttccctt cggtgccgga accctcccaa |
| 2761 | ttcccctccc tggccccaca tccccaaccc tgctgctgga gtgctctcgc aaacccctgc |
| 2821 | tgtcgcctgg aaaaaagtgc ccaagctgct gacgcaaaaa g |
| mouse espin isoform 1 amino acid sequence (NCBI Reference | |
| Sequence: NP_997570.1) | |
| Sequence ID No: 4 | |
| 1 | maleqalqaa rrgdldvlrs lhaagllgps lrdsldalpv hhaarsgklh clrylveeva |
| 61 | lpavsrarng atpandaaat gylsclqwll tqggcrvqek dnsgatvlhl aarfghpdvv |
| 121 | kwllyqggan saittdtgal pihyaaakgd lpslkllvgh ypegvnaqtn ngatplylac |
| 181 | qeghlevtky lvqecsadph lraqdgmtpl haaaqmghnp vlvwlvsfad vsfseqdhdg |
| 241 | atamhfaasr ghtkvlswll lhgaeisqdl wggtplhdaa engeleccqi lavngagldv |
| 301 | rdhdgytaad laefnghthc srylrtvqtl slehrvlsrd qsmdleakql dsgmsspntt |
| 361 | msvqpmtfdl gsptstfsny dscssshsss kgqrsnrgip garaadlqsy mdmlnpeksl |
| 421 | prgklgkpsp ppppppppps fpppppptgt qppppppgyp apnppvglhl nniymqtknk |
| 481 | lrhvevdslk epkvelndqf aqpssgdghs glhrqdsgll rqdsellhrq ellrhstglr |
| 541 | rqdsdrkqrs fskqpstgdy yrqlgrspge plaarpgmah seeaallpgn hvhngcsads |
| 601 | kasrelpppp pppplpeals spppapplpi egagaacgqr rsssstgkvr vlrhrkstks |
| 661 | fnmmsptgdn sellaeikag kslkptpqsk glttvfsgsg qpasqpespq plvspapsrt |
| 721 | rsptppasgs qpllngsvvp appatpapgv hldvealipt ldeqgrpipe wkrqvmvrkl |
| 781 | qqkmqeeeeq rrkeeeeear laslpawrrd ilrkkleeer eqkrkeeerq kleeiqrake |
| 841 | qseklrtlgy deaklapwqr qvilkkgeip k |
| Mus musculus espin (Espn), transcript variant 4, mRNA (NCBI | |
| Reference Sequence: NM_207689.2) | |
| Sequence ID No: 5 | |
| 1 | agagaggagg aggaggagca ggcttgccgg tagcagggac agcatgggca atagcttgga |
| 61 | acaccgagtc ttgtcccggg atcaatccat ggacctggag gcaaagcagc tggactcggg |
| 121 | tatgtcctcg cccaacacca ccatgtcggt ccagccaatg acctttgacc tgggctcgcc |
| 181 | tactagcacg ttctccaact atgactcctg ctcctccagt cactccagca gcaaggggca |
| 241 | gcgatcgaat cgagggattc caggtgcaag agctgcagac ttacagagct acatggacat |
| 301 | gctgaaccca gagaagagct tgcctcgggg caagctaggg aagccttccc cgccaccacc |
| 361 | tccaccacca ccaccaccaa gcttcccgcc acccccacca cccacaggca cccagccgcc |
| 421 | cccacctcca ccaggctacc cagctcccaa tccccctgtg ggactgcatc tgaataacat |
| 481 | ttacatgcag accaagaaca agcttcgcca tgtggaggtg gactcgctca aggaggcggc |
| 541 | gctgctcccc gggaaccacg tgcacaacgg ctgctcagcg gactccaaag cgtccaggga |
| 601 | gctgccgccg ccaccgccgc cgccgccgct gcccgaggcc ctgagttcgc cgccgcccgc |
| 661 | cccacctctg cccatcgagg gcgcgggcgc agcctgcggg cagcgtcgtt cctcgtcttc |
| 721 | tactggcaaa gtgagagtcc tgagacacag gaagagcacc aaatctttca acatgatgtc |
| 781 | cccaacgggt gataactcag agcttctggc tgagataaag gcgggcaaga gcctgaagcc |
| 841 | gacaccgcag agcaaggggc tgacaaccgt gttctcaggc agtgggcagc cagcctccca |
| 901 | gcctgagtca ccgcagcctc tggtgtcacc tgcgccatct cggactcgga gccccacccc |
| 961 | gccagcctct gggtctcagc cactgctcaa tggcagtgtg gtgccggcac cacctgccac |
| 1021 | cccggcacct ggagtccatc tggatgtgga ggccctcatt cccactcttg atgagcaggg |
| 1081 | ccggcccatc ccggagtgga agcgccaggt gatggtccgc aagctgcagc agaagatgca |
| 1141 | ggaggaagag gagcagcgga ggaaggagga agaggaggag gcccggctcg ccagcctgcc |
| 1201 | tgcctggaga cgagacattc ttcggaagaa gctggaggag gagagggagc agaagcgaaa |
| 1261 | agaggaggag cggcaaaagc tggaggaaat acagagggcg aaagaacagt cggagaagct |
| 1321 | gcggacacta ggctacgacg aagccaagct cgcgccctgg cagcgacagg tcatcttgaa |
| 1381 | gaagggggag atccctaagt aataggagtc tctcggcttc ttgcgtgtag cctcacaagt |
| 1441 | tctgagagat ggggagcggc ccccagcccc cgccccatcc tgtcaggttg gtgcggaagg |
| 1501 | gctaggatcc ttgccgccct tcccttcggt gccggaaccc tcccaattcc cctccctggc |
| 1561 | cccacatccc caaccctgct gctggagtgc tctcgcaaac ccctgctgtc gcctggaaaa |
| 1621 | aagtgcccaa gctgctgacg caaaaag |
| mouse espin isoform 4 amino acid sequence (NCBI Reference | |
| Sequence: NP_997572.1) | |
| Sequence ID No: 6 | |
| 1 | mgnslehrvl srdqsmdlea kqldsgmssp nttmsvqpmt fdlgsptstf snydscsssh |
| 61 | ssskgqrsnr gipgaraadl qsymdmlnpe kslprgklgk pspppppppp ppsfpppppp |
| 121 | tgtqpppppp gypapnppvg lhlnniymqt knklrhvevd slkeaallpg nhvhngcsad |
| 181 | skasrelppp ppppplpeal sspppapplp iegagaacgq rrsssstgkv rvlrhrkstk |
| 241 | sfnmmsptgd nsellaeika gkslkptpqs kglttvfsgs gqpasqpesp qplvspapsr |
| 301 | trsptppasg sqpllngsvv pappatpapg vhldvealip tldeqgrpip ewkrqvmvrk |
| 361 | lqqkmqeeee qrrkeeeeea rlaslpawrr dilrkkleee reqkrkeeer qkleeiqrak |
| 421 | eqseklrtlg ydeaklapwq rqvilkkgei pk |
| rat espin (Espn), transcript variant 1, mRNA (NCBI Reference | |
| Sequence: NM_019622.2) | |
| Sequence ID No: 7 | |
| 1 | gccgagggtg acaccatggc cctggaacag gcgatgcagg cggcacggcg gggcgacctg |
| 61 | gacgtgctga ggtccctgca cgccgccggc ctgctggggc cttctctgcg cgacccgcta |
| 121 | gacgccctgc cggtgcacca tgcggcccgc tcaggcaagc tgcactgttt gcgctacttg |
| 181 | gtggaggagg ttgccctccc agctgtgtcc cgcgcgcgca acggcgccac accagcccat |
| 241 | gatgccgctg ccacgggcta cctctcttgc ctgcagtggc tgctcacaca gggtggctgc |
| 301 | agggtgcagg aaaaagataa ctctggtgcc acggtcctgc acctggctgc ccgctttggc |
| 361 | cacccggacg tggtgaactg gctgctgtac cagggcggtg cgaactctgc catcaccaca |
| 421 | gacacgggcg ccctgcctat ccactatgct gccgccaaag gagatctccc ctccatgaag |
| 481 | cttcttgtcg ggcactaccc tgagggagtg aatgcccaaa ccaacaacgg tgccacgccc |
| 541 | ctgtacctgg cgtgccagga gggccacctg gaagtgacga agtaccttgt gcaggagtgc |
| 601 | agtgcagatc cgcacctgcg cgcccaagac ggcatgaccc ccctgcatgc cgcggcgcag |
| 661 | atgggccaca acccagtcct ggtgtggctg gtgagctttg cggacgtgag cttttcggag |
| 721 | caggaccacg acggcgccac agccatgcac tttgcagcca gccgcggcca caccaaagtg |
| 781 | ctcagctggc tcctgctgca cggcgcagag atctcccagg acctgtgggg cgggaccccg |
| 841 | ctgcatgatg ctgctgagaa cggggaactg gagtgctgcc agatcctcgc ggtgaatggt |
| 901 | gcggggctgg acgtccgcga ccacgatggg tatacggctg cggacctggc tgatttcaat |
| 961 | ggccacaccc actgctcccg ctacctacgt acggtgcaaa ccctgagctt ggaacaccga |
| 1021 | gtcctgtccc gggatccatc catggacctg gaggcaaagc agccggactc aggcatgtct |
| 1081 | tcacccaaca ccaccatgtc ggtccagccg ccgaactttg accttggctc acccaccagc |
| 1141 | accctctcca actatgactc ctgctcctcc agccattcca gcagcaaggg tcagcgctct |
| 1201 | actcgaggtg ccagatcctc agacttacag agctacatgg acatgctgaa cccagagcct |
| 1261 | cggagcaagc aagggaagcc ttcatctcta ccaccaccgc caccaccaag cttccctcca |
| 1321 | ccaccacccc caggcacaca gctgccccca cctccaccag gctacccagc tcccaatccc |
| 1381 | cctgtggggc tgcatttgga taacatttac atgcagacca agaacaaact ccgccacgtg |
| 1441 | gaggtggact ccctcaagaa ggagccgagc tccggcgacg gctactcggg gctacgcagg |
| 1501 | caggattccg ggctgctcag gcaggattcg gagctgctgc tcaggcacaa caccggactg |
| 1561 | cgcaggcagg actccgaccg caaacagcgt tcattcagta aacagcccag cacgggggac |
| 1621 | tactaccgcc agctgggccg cagcccgggg gagccgctgg ccgcacgccc gggcatggcc |
| 1681 | cacagcgagg aggcggcgct gctccccggg aaccacgtgc acaacggctg ctccgcggac |
| 1741 | tccaaagcgt ccagggagct gccgccgcca ccgccgccgc cgcccctgcc cgaggccctg |
| 1801 | agttcgccgc cacccgcccc acctctgccc atcgagggcg cgggcgcagc ctgcggacag |
| 1861 | cggcgttcct catcttctac tggcagcacc aaatctttca acatgatgtc cccaacgggt |
| 1921 | gacaactcgg agttgctggc tgaaataaaa gcaggcaaga gtctgaaacc aacaccgcag |
| 1981 | agcaaggggc tgacaacagt gttctcaggc agcgggcagc cagcctccca gcctgagtca |
| 2041 | ccacagcctg cggtgtcacc tgggccatct cgggcccgga gccccacccc accagcctct |
| 2101 | gggcctcagc cactgctcaa tggcagcata gtaccggcac cacctgccac cctagcacca |
| 2161 | ggagtgcatc tggatgtgga ggccctcatc cccacacttg atgagcaggg ccggcccatc |
| 2221 | ccggagtgga agcgccaggt gatggtccgg aagctgcagc agaaaatgca ggaggaagag |
| 2281 | gagcagcgga ggaaggaaga agaggaggag gcccggctgg ccagcctgcc cgcctggaga |
| 2341 | cgagacattc ttaggaagaa gctggaggag gagagggagc agaagcgaaa agaggaggag |
| 2401 | cggcagaagc tggaggaaat ccagagggcg aaagaacagt cggagaagct gcggacacta |
| 2461 | ggatacgacg aggccaagct cgcgccctgg cagcgacagg tcatcttgaa gaagggggag |
| 2521 | atccctaagt aataggagtc tcggcctctt gcgtgtagcc tcacgagctc tgagaaatgg |
| 2581 | ggagcggccc ccagcccccg ccccatcctg tcaggttggt gcggaagggt gggagccttg |
| 2641 | agcccttccc ttcggtgcct gccggaatct tccctattcc cctccctggc cccacatccc |
| 2701 | caaccctgct gctggagtgc tctcgcgaac ccctgctgtc gcctggaaaa aaaaagtgcc |
| 2761 | caggctgctg acgcaaaaag aaaaaaagag aaagaaaaaa gatgctcatt tgcatgctca |
| 2821 | cttacatata tttgcatgtt caccgaccca gccggagctc gccccagcct cgtgggtggt |
| 2881 | gacttttcct gcagggcgca cgccctgctg cagccccctc ccccgcagcc cggaacccac |
| 2941 | attgctggcg catcctgggt ggaggcagac cccgagctcg gggaaggggt tttcctccct |
| 3001 | ccacgaccca gatctgtgct ggccaaagtt gctgcgtttc ttatcccctg cgagggtttc |
| 3061 | ctctcagtca tttgtttacc agaaaggatg aaacttgcct gtaggggcgg agctgtcgct |
| 3121 | cctgaggggc cccaccttta cacgtaccac cccctccccc cgccccagtt acgccttcca |
| 3181 | agttcccagt caggccctta gcttccctga ccctttcact gcctcaatgg tataagtgga |
| 3241 | gggccaggga tcccgcaatt gtacctgctc ctcaccctat cccccacccc cttccttccc |
| 3301 | ccattaaacc tctatccgcc aa |
| rat espin isoform 1 amino acid sequence (NCBI Reference | |
| Sequence: NP_062568.2) | |
| Sequence ID No: 8 | |
| 1 | maleqamqaa rrgdldvlrs lhaagllgps lrdpldalpv hhaarsgklh clrylveeva |
| 61 | lpavsrarng atpahdaaat gylsclqwll tqggcrvqek dnsgatvlhl aarfghpdvv |
| 121 | nwllyqggan saittdtgal pihyaaakgd lpsmkllvgh ypegvnaqtn ngatplylac |
| 181 | qeghlevtky lvqecsadph lraqdgmtpl haaaqmghnp vlvwlvsfad vsfseqdhdg |
| 241 | atamhfaasr ghtkvlswll lhgaeisqdl wggtplhdaa engeleccqi lavngagldv |
| 301 | rdhdgytaad ladfnghthc srylrtvqtl slehrvlsrd psmdleakqp dsgmsspntt |
| 361 | msvqppnfdl gsptstlsny dscssshsss kgqrstrgar ssdlqsymdm lnpeprskqg |
| 421 | kpsslppppp psfppppppg tqlpppppgy papnppvglh ldniymqtkn klrhvevdsl |
| 481 | kkepssgdgy sglrrqdsgl lrqdselllr hntglrrqds drkqrsfskq pstgdyyrql |
| 541 | grspgeplaa rpgmahseea allpgnhvhn gcsadskasr elpppppppp lpealssppp |
| 601 | applpiegag aacgqrrsss stgstksfnm msptgdnsel laeikagksl kptpqskglt |
| 661 | tvfsgsgqpa sqpespqpav spgpsrarsp tppasgpqpl lngsivpapp atlapgvhld |
| 721 | vealiptlde qgrpipewkr qvmvrklqqk mqeeeeqrrk eeeeearlas lpawrrdilr |
| 781 | kkleeereqk rkeeerqkle eiqrakeqse klrtlgydea klapwqrqvi lkkgeipk |
| rat espin (Espn), transcript variant 4, mRNA (GenBank: | |
| AF540947.1; GI:28822427) | |
| Sequence ID No: 9 | |
| 1 | atgggtgata gcttggaaca ccgagtcctg tcccgggatc catccatgga cctggaggca |
| 61 | aagcagccgg actcaggcat gtcttcaccc aacaccacca tgtcggtcca gccgccgaac |
| 121 | tttgaccttg gctcacccac cagcaccctc tccaactatg actcctgctc ctccagccat |
| 181 | tccagcagca agggtcagcg ctctactcga ggtgccagat cctcagactt acagagctac |
| 241 | atggacatgc tgaacccaga gcctcggagc aagcaaggga agccttcatc tctaccacca |
| 301 | ccgccaccac caagcttccc tccaccacca cccccaggca cacagctgcc cccacctcca |
| 361 | ccaggctacc cagctcccaa tccccctgtg gggctgcatt tggataacat ttacatgcag |
| 421 | accaagaaca aactccgcca cgtggaggtg gactccctca agaaggagcc gagctccggc |
| 481 | gacggctact cggggctacg caggcaggat tccgggctgc tcaggcagga ttcggagctg |
| 541 | ctgctcaggc acaacaccgg actgcgcagg caggactccg accgcaaaca gcgttcattc |
| 601 | agtaaacagc ccagcacggg ggactactac cgccagctgg gccgcagccc gggggagccg |
| 661 | ctggccgcac gcccgggcat ggcccacagc gaggaggcgg cgctgctccc cgggaaccac |
| 721 | gtgcacaacg gctgctccgc ggactccaaa gcgtccaggg agctgccgcc gccaccgccg |
| 781 | ccgccgcccc tgcccgaggc cctgagttcg ccgccacccg ccccacctct gcccatcgag |
| 841 | ggcgcgggcg cagcctgcgg acagcggcgt tcctcatctt ctactggcaa agtgagaatc |
| 901 | ctgagacaca ggaagagcac caaatctttc aacatgatgt ccccaacggg tgacaactcg |
| 961 | gagttgctgg ctgaaataaa agcaggcaag agtctgaaac caacaccgca gagcaagggg |
| 1021 | ctgacaacag tgttctcagg cagcgggcag ccagcctccc agcctgagtc accacagcct |
| 1081 | gcggtgtcac ctgggccatc tcgggcccgg agccccaccc caccagcctc tgggcctcag |
| 1141 | ccactgctca atggcagcat agtaccggca ccacctgcca ccctagcacc aggagtgcat |
| 1201 | ctggatgtgg aggccctcat ccccacactt gatgagcagg gccggcccat cccggagtgg |
| 1261 | aagcgccagg tgatggtccg gaagctgcag cagaaaatgc aggaggaaga ggagcagcgg |
| 1321 | aggaaggaag aagaggagga ggcccggctg gccagcctgc ccgcctggag acgagacatt |
| 1381 | cttaggaaga agctggagga ggagagggag cagaagcgaa aagaggagga gcggcagaag |
| 1441 | ctggaggaaa tccagagggc gaaagaacag tcggagaagc tgcggacact aggatacgac |
| 1501 | gaggccaagc tcgcgccctg gcagcgacag gtcatcttga agaaggggga gatccctaag |
| 1561 | taa |
| rat espin isoform 4 amino acid sequence (GenBank: | |
| AAO50331.1; GI:28822428) | |
| Sequence ID No: 10 | |
| 1 | mgdslehrvl srdpsmdlea kqpdsgmssp nttmsvqppn fdlgsptstl snydscsssh |
| 61 | ssskgqrstr garssdlqsy mdmlnpeprs kqgkpsslpp ppppsfpppp ppgtqlpppp |
| 121 | pgypapnppv glhldniymq tknklrhvev dslkkepssg dgysglrrqd sgllrqdsel |
| 181 | llrhntglrr qdsdrkqrsf skqpstgdyy rqlgrspgep laarpgmahs eeaallpgnh |
| 241 | vhngcsadsk asrelppppp ppplpealss pppapplpie gagaacgqrr sssstgkvri |
| 301 | lrhrkstksf nmmsptgdns ellaeikagk slkptpqskg lttvfsgsgq pasqpespqp |
| 361 | avspgpsrar sptppasgpq pllngsivpa ppatlapgvh ldvealiptl deqgrpipew |
| 421 | krqvmvrklq qkmqeeeeqr rkeeeeearl aslpawrrdi lrkkleeere qkrkeeerqk |
| 481 | leeiqrakeq seklrtlgyd eaklapwqrq vilkkgeipk |

What is claimed is:

1. A method of inducing stereocilia and/or stereociliary bundles in an inner ear auditory hair cell in a subject in need thereof comprising administering to and expressing within the hair cell in the inner ear a polynucleotide encoding espin isoform 1 (ESPN1).

2. The method of claim 1, wherein the subject is human.

3. A method of inducing stereocilia and/or stereociliary bundles in an inner ear auditory hair cell comprising administering to and expressing within the hair cell a polynucleotide encoding espin isoform 1 (ESPN1).

4. The method of claim 3, wherein the hair cell is in vivo.

5. The method of claim 3, wherein the hair cell is in vitro.

6. The method of any one of claims 1 to 5, wherein the hair cell is a damaged or regenerated hair cell.

7. The method of claim 6, wherein the hair cell has been damaged by exposure to an aminoglycoside.

8. The method of any one of claims 1 to 7, wherein the polynucleotide encoding ESPN1 is expressed under the control of a promoter heterologous to the polynucleotide.

9. The method of any one of claims 1 to 8, wherein the polynucleotide encoding ESPN1 is administered to the hair cell in a viral vector.

10. The method of claim 9, wherein the viral vector is from a virus selected from the group consisting of adenovirus, adeno-associated virus, lentivirus, and retrovirus.

11. The method of claim 10, wherein the viral vector is replication defective.

12. The method of any one of claims 1 to 8, wherein the polynucleotide encoding ESPN1 is administered to the hair cell in a plasmid vector.

13. The method of any one of claims 1 to 12, wherein the polynucleotide encoding ESPN1 has at least 60% sequence identity to one or more of SEQ ID NOs: 1, 3, 5, 7 or 9.

14. The method of any one of claims 1 to 13, further comprising administering to the hair cell a polynucleotide encoding ATOH1 (atonal bHLH transcription factor 1).

15. The method of any one of claims 1 to 14, further comprising administering to the hair cell a γ-secretase inhibitor.

16. The method of claim 15, wherein the γ-secretase inhibitor is DAPT (N-[N-[(3,5-Difluorophenyl)acetyl]-L-alanyl]-L-phenylglycinetert-butyl).

17. The method of any one of claims 1 to 16, wherein the induced stereociliary bundles comprise a stair-step arrangement and a single, central kinocilium.

18. The method of any one of claims 1 to 17, wherein the induced stereocilia in a stereociliary bundle are linked at their tops.

19. The method of any one of claims 1 to 18, wherein the induced stereociliary bundles exhibit functional mechanoelectrical transduction channels.

20. A method of inducing stereocilia and/or stereociliary bundles in an inner ear auditory hair cell in a subject in need thereof comprising administering to the hair cell in the inner ear an ESPN1 polypeptide.

21. The method of claim 20, wherein the subject is human.

22. A method of inducing stereocilia and/or stereociliary bundles in an inner ear auditory hair cell comprising administering to the hair cell an ESPN1 polypeptide.

23. The method of claim 22, wherein the hair cell is in vivo.

24. The method of claim 22, wherein the hair cell is in vitro.

25. The method of any one of claims 20 to 24, wherein the hair cell is a damaged or regenerated hair cell.

26. The method of claim 25, wherein the hair cell has been damaged by exposure to an aminoglycoside.

27. The method of any one of claims 20 to 26, wherein the ESPN1 polypeptide is administered in a nanoparticle, a liposome and/or a microparticle.

28. The method of any one of claims 20 to 27, wherein the ESPN1 polypeptide has at least 60% sequence identity to one or more of SEQ ID NOs: 2, 4, 6, 8 or 10.

29. The method of any one of claims 20 to 28, further comprising administering to the hair cell a polynucleotide encoding ATOH1 (atonal bHLH transcription factor 1).

30. The method of any one of claims 20 to 29, further comprising administering to the hair cell a γ-secretase inhibitor.

31. The method of claim 30, wherein the y-secretase inhibitor is DAPT (N-[N-[(3,5-Difluorophenyl)acetyl]-L-alanyl]-L-phenylglycinetert-butyl).

32. The method of any one of claims 20 to 31, wherein the induced stereociliary bundles comprise a stair-step arrangement and a single, central kinocilium.

33. The method of any one of claims 20 to 32, wherein the induced stereocilia in a stereociliary bundle are linked at their tops.

34. The method of any one of claims 20 to 33, wherein the induced stereociliary bundles exhibit functional mechanoelectrical transduction channels.

35. An otic solution comprising a polynucleotide encoding espin isoform 1 (ESPN1).

36. The otic solution of claim 35, wherein the polynucleotide is in a viral vector or a plasmid vector.

37. The otic solution of any one of claims 35 to 36, wherein the viral vector is from a virus selected from the group consisting of adenovirus, adeno-associated virus, lentivirus, and retrovirus.

38. The otic solution of claim 37, wherein the viral vector is replication defective.

39. The otic solution of any one of claims 35 to 38, wherein the polynucleotide encoding ESPN1 has at least 60% sequence identity to one or more of SEQ ID NOs: 1, 3, 5, 7 or 9.

40. An otic solution comprising an ESPN1 polypeptide.

41. The otic solution of claim 40, wherein the ESPN1 polypeptide has at least 60% sequence identity to one or more of SEQ ID NOs: 2, 4, 6, 8 or 10.
